SEQUENCE LISTING (2 sequences)

SEQ ID NO: 1 - protein, 10 aa, Artificial
  Synthetic Melan-A/MART-1 optimized
ELAGIGILTV

SEQ ID NO: 2 - protein, 10 aa, Artificial
  Synthetic Melan-A/MART-1
EAAGIGILTV

(19) United States
(12) Patent Application Publication
(10) Pub. No.: US 2016/0341718 A1
(43) Pub. Date: Nov. 24, 2016

(54) METHODS FOR TESTING T CELL PRIMING EFFICACY IN A SUBJECT

(71) Applicants: INSERM (INSTITUT NATIONAL DE LA SANTÉ ET DE LA RECHERCHE MÉDICALE), Paris (FR); UNIVERSITÉ PARIS - SUD, Orsay (FR)

(72) Inventors: Roberto MALLONE, Paris (FR); Victor APPAY, Paris (FR); Anna LISSINA, Paris (FR)

(21) Appl. No.: 15/112,475

(22) PCT Filed: Jan. 20, 2015
(86) PCT No.: PCT/EP2015/050926

§ 371 Date: Jul. 19, 2016

(30) Foreign Application Priority Data
Jan. 21, 2014 (EP) 14305080.5

(51) Int. Cl. G01N 33/50; A61K 39/00; C12N 5/0783; C12N 5/095; C12N 5/0784

(52) U.S. Cl. CPC G01N 33/505; C12N 5/0695; C12N 5/0639; C12N 5/0636; A61K 39/0011; C12N 2501/22; C12N 2501/2304; C12N 2501/999; C12N 2501/2301; C12N 2501/2307; C12N 2501/24; C12N 2501/25; C12N 2501/06; C12N 2500/84

(57) ABSTRACT

The present invention relates to methods for testing T cell priming efficacy in a subject. In particular the present invention relates to an in vitro method for testing T cell priming efficacy in a subject comprising the steps of a) providing sample from the subject, b) culturing the sample in a medium which induces the differentiation of dendritic cells, c) maturing the dendritic cells obtained at step a) in presence of an amount of at least one antigen and an amount of at least one cytokine or ligand suitable for the activation of a pathogen recognition receptor, d) priming and expanding the T cells present in the sample and e) analyzing the func tionality of the primed T cells.

### FIELD OF THE INVENTION

The present invention relates to methods for testing T cell priming efficacy in a subject.

### BACKGROUND OF THE INVENTION

T cells are major actors of our immune system. Owing to their potent effector functions, T cells play a key role in the fight against foreign pathogens and tumor development in humans (Appay et al., 2008. Nat Med 14, 623-8). Understanding the principles of their efficacy or the attributes of effective memory T cells is key in immunology. Notably, T cells participate to the establishment of immunological memory, the founding principle of vaccinology. Much effort has thus been devoted to the development of T cell based vaccines in infectious (e.g. HIV or HPV) or cancerous (e.g. melanoma) contexts. T cell based vaccines aim at inducing effective memory T cells from the pool of naive precursors present in vaccinees. However, this faces several challenges like: what is the best vaccine formulation (e.g. in terms of antigen and adjuvants) to effectively prime naive T cell precursors? Or which immunological parameters impact on T cell priming and thus vaccination efficacy in humans, for instance with age? Accordingly, methods for testing T cell priming efficacy are highly desirable.

Multiple parameters determine the fate of T cells upon priming with an antigen and their differentiation into effector/memory T cells. The nature and strength of the signals delivered to T cells and therefore the selection of certain antigen specific T cell repertoire depend on the type of antigen presenting cells (APCs) and signal received for maturation. These parameters can be directly influenced by the type of pathogen recognition receptors (PRR) engaged in APCs prior to T cell priming. APCs express an array of PRR that have diverse cellular localizations, structures and ligands. C-type lectins (e.g. Dectin-1, DC-SIGN) and Toll-like receptors (TLR) are transmembrane proteins that interact with glycoproteins or specific molecular structures such as bacterial deoxycytidyl-deoxyguanosin (CpG), respectively. Nod-like (NLR) and RIG-like receptors (RLR) are cytosolic proteins that bind bacterial peptidoglycans and viral RNA/DNA, respectively (Kawai and Akira. 2009. Int Immunol 21, 317-37). PRR engagement in APCs triggers cytokine/chemokine secretion and functional maturation of DC, which alter antigen uptake, processing and presentation, as well as co-stimulation abilities, required for optimal T cell activation and priming capacities. Overall, PRR, such as TLR, determine the nature of the signals delivered to T cells and thus, T cell activation, TCR repertoire selection and clonal expansion. The discovery of PRR has opened new avenues for the development of safe and effective adjuvants. PRR ligands can be incorporated into adjuvants for vaccination in order to target specific APC populations, and influence the TCR threshold of activation during T cell priming (Malherbe et al. 2008. Immunity 28, 698-709; and Zhu et al. 2010. J Clin Invest 120, 607-16). Selecting the right PRR ligands in order to preferentially induce T cells endowed with high antigen sensitivity represents a major challenge in vaccinology.

Moreover, the naïve T cell precursor frequency as well as the cytokine and inflammatory environment can shape the T cell repertoire upon T cell priming and influence greatly the induction of an effective T cell response. In mice, precursor frequency can correlate with the magnitude of the primary T-cell response and memory cell immunodominance patterns (Obar et al. 2008. Immunity 28, 859-69; and Moon et al. 2007. Immunity 27, 203-13). Preservation of memory cells can also be independent of TCR signaling(Surh and Sprent, 2008). Memory T-cell maintenance is regulated by a combination of IL-7 and IL-15, which primarily support cell viability and basal homeostatic proliferation, respectively. However, we know that both the frequency of naïve T-cell precursors and the production capacity of these soluble factors can change widely with age or various inflammatory conditions (for instance associated with infections with viruses like HIV). These different factors represent as many variables to consider in vaccinology and integrate in complex analyses of T cell priming in humans.

### SUMMARY OF THE INVENTION

The present invention relates to methods for testing T cell priming efficacy in humans and applications thereof in vaccinology. In particular, the present invention is defined by the claims.

### DETAILED DESCRIPTION OF THE INVENTION

The present invention relates to a simple and original in vitro model for testing in humans the induction of effective T cell responses from naïve precursors. This method is particularly suitable for A) the identification and selection of the best ligands for pattern recognition receptors susceptible to be used as adjuvants to induce high quality T cells through vaccination in humans; and B) the study of immune parameters associated with declining immune competence and the prediction of response to vaccines in humans (e.g. aging, immune deficiencies, viral infections, cancer, transplantation . . . ). For instance, the inventors focus on individuals harboring the very common HLA-A2 allele who are known to present a remarkably large population of naïve CD8+ T cells reactive for Melan-A (Dutoit et al. 2002. J Exp Med 196, 207-16; and Zippelius et al. 2002. J Exp Med 195, 485-94), used here as model antigen to demonstrate proof of concept. They show that these cells can be effectively primed in vitro into large memory T cell populations using as few as 10⁶ total peripheral blood mononuclear cells (PBMC). This represents a unique setting to compare the qualitative attributes of antigen specific T cells induced upon priming in multiple conditions. The present method has great potential by providing highly relevant information on T cell priming capacity particularly in humans, related to vaccinology as well as different contexts (e.g. aging, immune deficiencies, viral infections, cancer, transplantation).

Accordingly a first object of the invention relates to an in vitro method for testing T cell priming efficacy in a subject comprising the steps of a) providing sample from the subject, b) culturing the sample in a medium which induces the differentiation of dendritic cells, c) maturing the dendritic cells obtained at step a) in presence of an amount of at least one antigen and an amount of at least one cytokine or ligand suitable for the activation of a pathogen recognition receptor, d) priming and expanding the T cells present in the sample and e) analyzing the polyfunctionality of the primed T cells.

In some embodiments, said subject is a human subject. Subjects may be male or female and may be of any age, including prenatal (i.e., in utero), neonatal, infant, juvenile, adolescent, adult, and geriatric subjects. The subject according to the invention can be a healthy subject or a subject suffering from a given disease (e.g. a subject suffering from a HIV infection).

In some embodiments, the method of the present invention is particularly suitable for testing CD4+ T cell priming efficacy.

In some embodiments, the method of the present invention is particularly suitable for testing CD8+ T cell priming efficacy.

In some embodiments, the subject harbors any HLA Class I (e.g., HLA-A, HLA-B, HLA-C, HLA-E, HLA-F, HLA-G, HLA-K, HLA-L) alleles and any HLA Class II (e.g., HLA-DP, HLA-DQ, HLA-DR, HLA-DM, HLA-DO) alleles. Methods for determining the HLA haplotype of the subject are well known in the art and may be performed on blood samples and involve use of HLA antibodies or molecular biology techniques. In some embodiments, the subject harbors the HLA-A2 (A*02:01) allele.

As used herein, the term “sample” refers to any sample that can be obtained from the subject so as to perfom the method of the present invention. In some embodiments, the sample is a tissue biopsy (e.g. tumor biopsy or transplant biopsy). In some embodiments, the sample is a PBMC sample obtained from the subject.

The term “PBMC” or “peripheral blood mononuclear cells” or “unfractionated PBMC”, as used herein, refers to whole PBMC, i.e. to a population of white blood cells having a round nucleus, which has not been enriched for a given sub-population. Cord blood mononuclear cells are further included in this definition. Typically, the PBMC sample according to the invention has not been subjected to a selection step to contain only adherent PBMC (which consist essentially of >90% monocytes) or non-adherent PBMC (which contain T cells, B cells, natural killer (NK) cells, NK T cells and DC precursors). A PBMC sample according to the invention therefore contains lymphocytes (B cells, T cells, NK cells, NKT cells), monocytes, and precursors thereof. Typically, these cells can be extracted from whole blood using Ficoll, a hydrophilic polysaccharide that separates layers of blood, with the PBMC forming a cell ring under a layer of plasma. Additionally, PBMC can be extracted from whole blood using a hypotonic lysis buffer which will preferentially lyse red blood cells. Such procedures are known to the expert in the art.

Any culture medium suitable for growth, survival and differentiation of PBMC may be used. Typically, it consists of a base medium containing nutrients (a source of carbon, aminoacids), a pH buffer and salts, which can be supplemented with serum of human or other origin and/or growth factors and/or antibiotics to which cytokines and the antigen are added. Typically, the base medium can be RPMI 1640, DMEM, IMDM, X-VIVO or AIM-V medium, all of which are commercially available standard media.

In some embodiments, the culture medium comprises an amount of Granulocyte/Macrophage Colony-Stimulating Factor (GM-CSF) and an amount of interleukin 4 (IL-4).

Typically, GM-CSF is used in an amount comprised between 1 and 10,000 U/ml, preferably between 10 and 5,000 U/ml, even more preferably at about 1,000 U/ml. GM-CSF can be obtained from a variety of sources. It may be purified or recombinant GM-CSF. GM-CSF is commercially available from different companies, for example R&D Systems or PeproTech.

Typically, IL-4 is used in an amount comprised between 0 and 10,000 U/ml, preferably between 10 and 1,000 U/ml, even more preferably at about 500 U/ml. IL-4 can be obtained from a variety of sources. It may be purified or recombinant IL-4. IL-4 is commercially available from different companies, for example R&D Systems or PeproTech.

In some embodiments, the culture medium comprises an amount of FMS-like tyrosine kinase 3 (Flt-3) ligand.

Typically, Flt-3 ligand is used in an amount comprised between 1 and 1,000 ng/ml, preferably between 10 and 100 ng/ml, even more preferably at about 50 ng/ml. Flt-3 ligand can be obtained from a variety of sources. It may be purified or recombinant Flt-3 ligand. Flt-3 ligand is commercially available from different companies, for example R&D Systems or PeproTech.

In some embodiments, the culture medium comprises an amount of IL-1β. As used herein the term “IL-1β” has its general meaning in the art and refers to interleukin-1β. Typically, IL-1beta is used in an amount comprised between 0.1 and 1,000 ng/ml, preferably between 1 and 100 ng/ml, even more preferably at about 10 ng/ml. IL-1beta can be obtained from a variety of sources. It may be purified or recombinant IL-1beta. IL-1beta is commercially available from different companies, for example R&D Systems or PeproTech.

According to the invention, step b) is performed for an amount of time sufficient for enriching the PBMC sample in dendritic cells. Thus the step is carried out for an amount of time t(b) comprised between t(b)min and t(b)max. Typically, the minimal incubation for step b), t(b)min, can be about 12 hours, preferably about 16 hours, even more preferably about 18 hours, about 19 hours, about 20 hours, about 21 hours, about 22 hours, about 23 hours, even more preferably about 24 hours. Typically, the maximum incubation for step b), t(b)max can be about 2 days, even more preferably about 1 day. In a preferred embodiment, step b) is carried out for an amount of time t(b) of about 24 hours.

As used herein the term “cytokine” has its general meaning in the art. Typically, examples of cytokines include lymphokines, interleukins, and chemokines

As used herein the term “interleukin” has its general meaning in the art and refers to any interleukin (e.g., IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, and IL-27) polypeptide. In some embodiments, the cytokine is selected from the group consisting of Interleukin 1 (IL-1), Interleukin 2 (IL-2), Interleukin 3 (IL-3), Interleukin 4 (IL-4), Interleukin 5 (IL-5), Interleukin 6 (IL-6), Interleukin 7 (IL-7), Interleukin 8 (IL-8), Interleukin 9 (IL-9), Interleukin 10 (IL-10), Interleukin 11 (IL-11), Interleukin 12 (IL-12), Interleukin 13 (IL-13), Interleukin 15 (IL-15), and Interleukin 17 (IL-17) polypeptides. In some embodiments, the interleukin is an inflammatory interleukin. In some embodiments, the interleukin is IL-1beta. Typically, IL-1beta is used in an amount comprised between 0.1 and 1,000 ng/ml, preferably between 1 and 100 ng/ml, even more preferably at about 10 ng/ml. IL-1beta can be obtained from a variety of sources. It may be purified or recombinant IL-1beta. IL-1beta is commercially available from different companies, for example R&D Systems or PeproTech. In some embodiments, the interleukin is IL-7. Typically, IL-7 is used in an amount comprised between 0.01 and 10 ng/ml, preferably between 0.1 and 1 ng/ml, even more preferably at about 0.5 ng/ml. IL-7 can be obtained from a variety of sources. It may be purified or recombinant IL-7. IL-7 is commercially available from different companies, for example R&D Systems or PeproTech.

As used herein the term “interferon” or “IFN” as used herein means the family of highly homologous species-specific proteins that inhibit viral replication and cellular proliferation and modulate immune response. Human interferons are grouped into two classes; Type I, including alpha and beta-interferon, and Type II, which is represented by gamma-interferon only. Recombinant forms of each group have been developed and are commercially available. In some embodiments, the interferon polypeptide is an interferon-alpha (IFN-alpha) polypeptide, an interferon-beta (IFN-β) polypeptide, or an interferon-gamma (IFN-gamma) polypeptide. In some embodiments, the interferon is an interferon-alpha (IFN-alpha) polypeptide. Typically, IFN-alpha is used in an amount comprised between 1 and 10,000 U/ml, preferably between 10 and 5,000 U/ml, even more preferably at about 1,000 U/ml. In a preferred embodiment, IFN-alpha is IFN-alpha2a. IFN-alpha can be obtained from a variety of sources. It may be purified or recombinant IFN-alpha. IFN-alpha is commercially available from different companies, for example Roche (Roferon-A), R&D Systems or PeproTech.

In some embodiments, the cytokine is TNF-alpha. Typically, TNF-alpha is used in an amount comprised between 1 and 10,000 U/ml, preferably between 10 and 5,000 U/ml, even more preferably at about 1,000 U/ml. TNF-alpha can be obtained from a variety of sources. It may be purified or recombinant TNF-alpha. TNF-alpha is commercially available from different companies, for example R&D Systems or PeproTech.

As used herein the term “pathogen recognition receptor” or “PRR” has its general meaning in the art and refers to a class of receptors expressed by cells of the innate immune system (including DCs, macrophages, mast cells and neutrophils) to identify pathogen-associated molecular patterns (PAMPs), which are associated with microbial pathogens or cellular stress, as well as damage-associated molecular patterns (DAMPs), which are associated with cell components released during cell damage. PPRs include membrane-bound PRRs (e.g. Receptor kinases, Toll-like receptors (TLR), C-type lectin Receptors) and cytoplasmic PRRs (e.g. NOD-like receptors (NLR), or RIG-I-like receptors).

In some embodiments, ligand that is suitable for the activation of a pathogen recognition receptor is a TLR agonist.

As used herein the term “Toll like receptor (TLR)” has its general meaning in the art and describes a member of the Toll-like receptor family of proteins or a fragment thereof that senses a microbial product and/or initiates an innate or an adaptive immune response. Toll-like receptors include TLR1, TLR2, TLR3, TLR4, TLR5, TLR6, TLR7, TLR 8, TLR9, TLR10, TR11 and TLR12. The term “agonist” as used herein in referring to a TLR activating molecule, means a molecule that activates a TLR signaling pathway. As discussed above, a TLR signaling pathway is an intracellular signal transduction pathway employed by a particular TLR that can be activated by the TLR agonist. Common intracellular pathways are employed by TLRs and include, for example, NF-κB, Jun N-terminal kinase and mitogen-activated protein kinase. The TLR agonism for a particular compound may be assessed in any suitable manner. For example, assays for detecting TLR agonism of test compounds are described, for example, in U.S. Provisional Patent Application Ser. No. 60/432,650, filed Dec. 11, 2002, and recombinant cell lines suitable for use in such assays are described, for example, in U.S. Provisional Patent Application Ser. No. 60/432,651, filed Dec. 11, 2002.

In one embodiment, the TLR agonist is selected from the group consisting of TLR1, TLR2, TLR3, TLR4, TLR5, TLR6, TLR7, TLR8, TLR9, TLR10, TLR11, TLR12, or TLR13 agonists. TLR agonists are well known in the art (see e.g. Baxevanis C N, Voutsas I F, Tsitsilonis O E. Toll-like receptor agonists: current status and future perspective on their utility as adjuvants in improving anticancer vaccination strategies. Immunotherapy, 2013 May; 5(5):497-511. doi: 10.2217/imt.13.24; Shaherin Basith, Balachandran Manavalan, Gwang Lee, Sang Geon Kim, Sangdun Choi Toll-like receptor modulators: a patent review (2006-2010) Expert Opinion on Therapeutic Patents June 2011, Vol. 21, No. 6, Pages 927-944; 20. Heather L. Davis Chapter 26: TLR9 Agonists for Immune Enhancement of Vaccines, New Generation Vaccines, Fourth Edition; Jory R Baldridge, Patrick McGowan, Jay T Evans, Christopher Cluff, Sally Mossman, David Johnson, David Persing Taking a Toll on human disease: Toll-like receptor 4 agonists as vaccine adjuvants and monotherapeutic agents Expert Opinion on Biological Therapy July 2004, Vol. 4, No. 7, Pages 1129-1138.).

In one embodiment, the TLR agonist is a TLR1 agonist. Examples of TLR1 agonists include tri-acylated lipopeptides (LPs); phenol-soluble modulin; Mycobacterium tuberculosis LP; S-(2,3-bis(palmitoyloxy)-(2-RS)-propyl)-N-palmitoyl-(R)-Cys-(S)-Ser-(S)-Lys(4)-OH, trihydrochloride (Pam3Cys) LP which mimics the acetylated amino terminus of a bacterial lipoprotein and OspA LP from Borrelia burgdorferi.

In one embodiment, the TLR agonist is a TLR2 agonist. For example, the TLR2 agonist consists of a flagellin modification protein FImB of Caulobacter crescentus; Bacterial Type III secretion system protein; invasin protein of Salmonella; Type 4 fimbrial biogenesis protein (PiIX) of Pseudomonas; Salmonella SciJ protein; putative integral membrane protein of Streptomyces; membrane protein of Pseudomonas; adhesin of Bordetella pertusis; peptidase B of Vibrio cholerae; virulence sensor protein of Bordetella; putative integral membrane protein of Neisseria meningitidis; fusion of flagellar biosynthesis proteins FIiR and FIhB of Clostridium; outer membrane protein (porin) of Acinetobacter; flagellar biosynthesis protein FIhF of Helicobacter; ompA related protein of Xanthomonas; omp2a porin of Brucella spp.; putative porin/fimbrial assembly protein (LHrE) of Salmonella; wbdKK of Salmonella; Glycosyltransferase involved in LPS biosynthesis; Salmonella putative permease. In one embodiment, the TLR2 agonist is selected form the group consisting of lipoprotein/lipopeptides (isolate from a variety of pathogens); peptidoglycan (isolated form Gram-positive bacteria); lipoteichoic acid (isolated from Gram-positive bacteria); lipoarabinomannan (isolated from mycobacteria); a phenol-soluble modulin (isolated from Staphylococcus epidermidis); glycoinositolphospholipids (isolated form Trypanosoma Cruzi); glycolipids (isolated from Treponema maltophilum); porins (isolated from Neisseria); zymosan (isolated from fungi) and atypical LPS (isolated form Leptospira interrogans and Porphyromonas gingivalis). The TLR2 agonist can also include at least one member selected from the group consisting of (see, PCT/US 2006/002906/WO 2006/083706; PCT/US 2006/003285/WO 2006/083792; PCTAJS 2006/041865; PCT/US 2006/042051). The TLR2 agonist can include at least a portion of a bacterial lipoprotein (BLP). The TLR2 agonist can be a bacterial lipoprotein, such as Pam2Cys (S-[2,3-bis(palmitoyloxy) propyl] cysteine), Pam3Cys ([Palmitoyl]-Cys((RS)-2,3-di(palmitoyloxy)-propyl cysteine) or Pseudomonas aeruginosa Oprl lipoprotein (Oprl). A bacterial lipoprotein that activates a TLR2 signaling pathway (a TLR2 agonist) is a bacterial protein that includes a palmitoleic acid (Omueti, K. O., et al, J. Biol. Chem. 280: 36616-36625 (2005)).

In one embodiment, the TLR agonist is a TLR3 agonist. For example, TLR3 agonists include naturally-occurring double-stranded RNA (dsRNA); synthetic ds RNA; and synthetic dsRNA analogs; and the like (Alexopoulou et al, 2001). An exemplary, non-limiting example of a synthetic dsRNA analog is Poly(I:C).

In one embodiment, the TLR agonist of the invention is a TLR4 agonist. Various TLR4 agonists are known in the art, including Monophosphoryl lipid A (MPLA), in the field also abbreviated to MPL, referring to naturally occurring components of bacterial lipopolysaccharide (LPS); refined detoxified endotoxin. For example, MPL is a derivative of lipid A from Salmonella minnesota R595 lipopolysaccharide (LPS or endotoxin). While LPS is a complex heterogeneous molecule, its lipid A portion is relatively similar across a wide variety of pathogenic strains of bacteria. MPL, used extensively as a vaccine adjuvant, has been shown to activate TLR4 (Martin M. et al., 2003. Infect Immun. 71(5):2498-507; Ogawa T. et al., 2002. Int Immunol. 14(11):1325-32). TLR4 agonists also include natural and synthetic derivatives of MPLA, such as 3-de-O-acylated monophosphoryl lipid A (3D-MPL), and MPLA adjuvants available from Corixa Corporation (Seattle, Wash.; see U.S. Pat. Nos. 4,436,727; 4,436,728; 4,987,237; 4,877,611; 4,866,034 and 4,912,094 for structures and methods of isolation and synthesis). A structure of MPLA is disclosed in U.S. Pat. No. 4,987,237. Non-toxic diphosphoryl lipid A (DPLA) may also be used, for example OM-174, a lipid A analogue of bacterial origin containing a triacyl motif linked to a diglucosamine diphosphate backbone. Another class of useful compounds are synthetic lipid A analogue pseudo-dipeptides derived from amino acids linked to three fatty acid chains (see for example EP 1242365), such as OM-197-MP-AC, a water soluble synthetic acylated pseudo-dipeptide (C55H107N4O12P). Non-toxic TLR4 agonists include also those disclosed in EP1091928, PCT/FR05/00575 or PCT/IB2006/050748. PCT/US2006/002906/WO 2006/083706; PCT/US 2006/003285/WO 2006/083792; PCT/US 2006/041865; PCT/US 2006/042051. TLR4 agonists also include synthetic compounds which signal through TLR4 other than those based on the lipid A core structure, for example an aminoalkyl glucosaminide 4-phosphate (see Evans J T et al. Expert Rev Vaccines. 2003 April; 2(2):219-29; or Persing et al. Trends Microbiol. 2002; 10(10 Suppl):532-7. Review). Other examples include those described in Orr M T, Duthie M S, Windish H P, Lucas E A, Guderian J A, Hudson T E, Shaverdian N, O'Donnell J, Desbien A L, Reed S G, Coler R N. MyD88 and TRIF synergistic interaction is required for TH1-cell polarization with a synthetic TLR4 agonist adjuvant. Eur J Immunol. 2013 May 29. doi: 10.1002/eji.201243124.; Lambert S L, Yang C F, Liu Z, Sweetwood R, Zhao J, Cheng L, Jin H, Woo J. Molecular and cellular response profiles induced by the TLR4 agonist-based adjuvant Glucopyranosyl Lipid A. PLoS One. 2012; 7(12):e51618. doi: 10.1371/journal.pone.0051618. Epub 2012 Dec. 28.

In one embodiment, the TLR agonist is a TLR5 agonist. Typically, the TLR5 agonist according to the invention is a flagellin polypeptide. As used herein, the term “flagellin” is intended to mean the flagellin contained in a variety of Gram-positive or Gram-negative bacterial species. Non-limiting sources of flagellins include but are not limited to Escherichia, e.g., E. coli, Enterobacter, Erwinia, Klebsiella, Proteus, Salmonella, e.g., Salmonella enterica serovar Typhimurium, Serratia, e.g., Serratia marcescans, and Shigella, as well as Bacilli such as B. subtilis and B. licheniformis, Pseudomonas such as P. aeruginosa, and Streptomyces. These examples are illustrative rather than limiting. The amino acid sequences and nucleotide sequences of flagellins are publically available in the NCBI Genbank, see for example Accession Nos. AAL20871, NP_310689, BAB58984, AAO85383, AAA27090, NP_461698, AAK58560, YP_001217666, YP_002151351, YP_001250079, AAA99807, CAL35450, AAN74969, and BAC44986. The flagellin sequences from these and other species are intended to be encompassed by the term flagellin as used herein. Therefore, the sequence differences between species are included within the meaning of the term. The term “flagellin polypeptide” is intended to a flagellin or a fragment thereof that retains the ability to bind and activate TLR5. Examples of flagellin polypeptides include but are not limited to those described in U.S. Pat. Nos. 6,585,980; 6,130,082; 5,888,810; 5,618,533; and 4,886,748; U.S. Patent Publication No. US 2003/0044429 A1; and in the International Patent Application Publications n° WO 2008097016 and WO 2009156405 which are incorporated by reference.

In one embodiment, the TLR agonist is a TLR7 agonist. For example, TLR7 agonists include, but are not limited to: imidazoquinoline-like molecules, imiquimod, resiquimod, gardiquimod, S-27609; and guanosine analogues such as loxoribine (7-allyl-7,8-dihydro-8-oxo-guanosine), 7-Thia-8-oxoguanosine and 7-deazaguanosine, UC-1V150, ANA975 (Anadys Pharmaceuticals), SM-360320 (Sumimoto), 3M-01 and 3M-03 (3M Pharmaceuticals) (see for example Gorden et al., J Immunology, 2005; Schön, Oncogene, 2008; Wu et al., PNAS 2007). TLR7 agonists include imidazoquinoline compounds; guanosine analogs; pyrimidinone compounds such as bropirimine and bropirimine analogs; and the like. Imidazoquinoline compounds that function as TLR7 ligands include, but are not limited to, imiquimod, (also known as Aldara, R-837, S-26308), and R-848 (also known as resiquimod, S-28463; having the chemical structure: 4-amino-2-ethoxymethyl-α, α.-dimethyl-1H-imidazol[4,5-c]quinoline-1-ethanol). Suitable imidazoquinoline agents include imidazoquinoline amines, imidazopyridine amines, 6,7-fused cycloalkylimidazopyridine amines, and 1,2 bridged imidazoquinoline amines.

In one embodiment, the TLR agonist is a TLR8 agonist. TLR8-selective agonists include those in U.S. Patent Publication 2004/0171086. Such TLR8 selective agonist compounds include, but are not limited to, the compounds shown in U.S. Patent Publication

No. 2004/0171086 that include N-{4-[4-amino-2-(2-methoxyethyl)-1H-imidazo[4,5-c]quinolin-1-yl]butyl}quinolin-3-carboxamide, N-{4-[4-amino-2-(2-methoxyethyl)-1H-imidazo[4,5-c]quinolin-1-yl]butyl}quinoxoline-2-carboxamide, and N-[4-(4-amino-2-propyl-1H-imidazo[4,5-c]quinolin-1-yl)butyl]morpholine-4-carboxamide. Other suitable TLR8-selective agonists include, but are not limited to, 2-propylthiazolo[4,5-c]quinolin-4-amine (U.S. Pat. No. 6,110,929); N1-[2-(4-amino-2-butyl-1H-imidazo[4,5-c] [1,5]naphthyridin-1-yl)ethyl]-2-amino-4-methylpentanamide (U.S. Pat. No. 6,194,425); N1-[4-(4-amino-1H-imidazo[4,5-c]quinolin-1-yl)butyl]-2-phenoxy-benzamide (U.S. Pat. No. 6,451,810); N1-[2-(4-amino-2-butyl-1H-imidazo[4,5-c]quinolin-1-yl)ethyl]-1-propanesulfonamide (U.S. Pat. No. 6,331,539); N-{2-[2-(4-amino-2-ethyl-1H-imidazo[4,5-c]quinolin-1-yl)ethyoxy]ethyl}-N′˜phenylurea (U.S. Patent Publication 2004/0171086); 1-{4-[3,5-dichlorophenyl)thio]butyl}-2-ethyl-1H-imidazo[4,5-c]quinolin-4˜amine (U.S. Patent Publication 2004/0171086); N-{2-[4-amino-2-(ethoxymethyl)-1H-imidazo[4,5-c]quinolin-1-yl]ethyl}-N′-(3-cyanophenyl)urea (WO 00/76518 and U.S. Patent Publication No. 2004/0171086); and 4-amino-α, α-dimethyl-2-methoxyethyl-1H-imidazo[4,5-c]quinoline-1-ethanol (U.S. Pat. No. 5,389,640). Included for use as TLR8-selective agonists are the compounds in U.S. Patent Publication No. 2004/0171086. Also suitable for use is the compound 2-propylthiazolo-4,5-c]quinolin-4-amine.

In a particular embodiment, the TLR agonist is a TLR9 agonist. Examples of TLR9 agonists (include nucleic acids comprising the sequence 5′-CG-3′ (a “CpG nucleic acid”), in certain aspects C is unmethylated. The terms “polynucleotide,” and “nucleic acid,” as used interchangeably herein in the context of TLR9 agonist molecules, refer to a polynucleotide of any length, and encompasses, inter alia, single- and double-stranded oligonucleotides (including deoxyribonucleotides, ribonucleotides, or both), modified oligonucleotides, and oligonucleosides, alone or as part of a larger nucleic acid construct, or as part of a conjugate with a non-nucleic acid molecule such as a polypeptide. Thus a TLR9 agonist may be, for example, single-stranded DNA (ssDNA), double-stranded DNA (dsDNA), single-stranded RNA (ssRNA) or double-stranded RNA (dsRNA). TLR9 agonists also encompass crude, detoxified bacterial (e.g., mycobacterial) RNA or DNA, as well as enriched plasmids enriched for a TLR9 agonist. In some embodiments, a “TLR9 agonist-enriched plasmid” refers to a linear or circular plasmid that comprises or is engineered to comprise a greater number of CpG motifs than normally found in mammalian DNA. Examples of non-limiting TLR9 agonist-enriched plasmids are described in Roman et al. (1997). In general, a TLR9 agonist used in a subject composition comprises at least one unmethylated CpG motif. In some embodiments, a TLR9 agonist comprises a central palindromic core sequence comprising at least one CpG sequence, where the central palindromic core sequence contains a phosphodiester backbone, and where the central palindromic core sequence is flanked on one or both sides by phosphorothioate backbone-containing polyguanosine sequences. In other embodiments, a TLR9 agonist comprises one or more TCG sequences at or near the 5′ end of the nucleic acid; and at least two additional CG dinucleotides. In some of these embodiments, the at least two additional CG dinucleotides are spaced three nucleotides, two nucleotides, or one nucleotide apart. In some of these embodiments, the at least two additional CG dinucleotides are contiguous with one another. In some of these embodiments, the TLR9 agonist comprises (TCG)n, where n=1 to 3, at the 5′ end of the nucleic acid. In other embodiments, the TLR9 agonist comprises (TCG)n, where n=1 to 3, and where the (TCG)n sequence is flanked by one nucleotide, two nucleotides, three nucleotides, four nucleotides, or five nucleotides, on the 5′ end of the (TCG)n sequence. A TLR9 agonist of the present invention includes, but is not limited to, any of those described in U.S. Pat. Nos. 6,194,388; 6,207,646; 6,239,116; 6,339,068; and 6,406,705, 6,426,334 and 6,476,000, and published US Patent Applications US 2002/0086295, US 2003/0212028, and US 2004/0248837.

In some embodiments, the ligand that is suitable for the activation of a pathogen recognition receptor is a NOD-like receptor ligand. The NOD-like receptor ligand can be without limitation selected from the group consisting of NOD1, NOD2, IPAF, Nalplb, and Cryopirin/Nalp3 ligand. The NOD-like receptor ligand is preferably meso-diaminopimelic acid, muramyl dipeptide or flagellin. Alternatively, the NOD-like receptor ligand is NOD1, NOD2, IPAF, Nalpl b or Cryopirin/Nalp3 ligand.

As used herein the term “antigen” has its general meaning in the art and refers to any compound (e.g. a peptide or polypeptide) that elicits and/or induces an immune response in a subject. The skilled person in the art will be able to select the appropriate antigen, depending on the desired CD8+ T cell stimulation.

In some embodiments, the antigen is a protein which can be obtained by recombinant DNA technology or by purification from different tissue or cell sources. Typically, said protein has a length higher than 10 aminoacids, preferably higher than 15 aminoacids, even more preferably higher than 20 aminoacids with no theoretical upper limit. Such proteins are not limited to natural ones, but also include modified proteins or chimeric constructs, obtained for example by post-translational modifications, by changing selected aminoacid sequences or by fusing portions of different proteins. In another embodiment of the invention, said antigen is a synthetic peptide. Typically, said synthetic peptide is 3-40 aminoacid-long, preferably 5-30 aminoacid-long, even more preferably 8-20 aminoacid-long. Synthetic peptides can be obtained by Fmoc biochemical procedures, large-scale multipin peptide synthesis, recombinant DNA technology or other suitable procedures. Such peptides are not limited to natural ones, but also include post-translationally modified aminoacids, modified peptides or chimeric peptides, obtained for example by changing selected aminoacid sequences or by fusing portions of different proteins.

In another embodiment of the invention, the antigen is a crude or partially purified tissue or cell preparation obtained by different biochemical procedures (e.g., fixation, lysis, subcellular fractionation, density gradient separation) known to the expert in the art.

Examples of antigens include viral antigens such as influenza viral antigens (e.g. hemagglutinin (HA) protein, matrix 2 (M2) protein, neuraminidase), respiratory syncitial virus (RSV) antigens (e.g. fusion protein, attachment glycoprotein), polio, papillomaviral (e.g. human papilloma virus (HPV), such as an E6 protein, E7 protein, L1 protein and L2 protein), Herpes Simplex, rabies virus and flavivirus viral antigens (e.g. Dengue viral antigens, West Nile viral antigens), hepatitis viral antigens including antigens from HBV and HC, human immunodeficiency virus (HIV) antigens (e.g. gag, pol or nef), herpesvirus (such as cytomegalovirus and Epstein-Barr virus) antigens (e.g. pp65, IE1, EBNA-1, BZLF-1) and adenovirus (AdV; e.g. A12, 18, 31; B3, 7, 11, 14, 16, 21, 34, 35, 50, 55; C1, 2, 5, 6, 57; D8, 9, 10, 13, 15, 17, 19, 20, 22, 23, 24, 25, 26, 27, 28, 29, 30, 32, 33, 36, 37, 38, 39, 42, 43, 44, 45, 46, 47, 48, 49, 51, 53, 54, 56; E4; F40, 41; G52) antigens (e.g. AdV hexon).

Other examples of antigens include bacterial antigens including those from Streptococcus pneumonia, Haemophilus influenza, Staphylococcus aureus, Clostridium difficile and enteric gram-negative pathogens including Escherichia, Salmonella, Shigella, Yersinia, Klebsiella, Pseudomonas, Enterobacter, Serratia, Proteus, B. anthracis, C. tetani, B. pertussis, S. pyogenes, S. aureus, N. meningitidis and Haemophilus influenzae type b.

Other examples of antigens include include fungal antigens including those from Candida spp., Aspergillus spp., Crytococcus neoformans, Coccidiodes spp., Histoplasma capsulatum, Pneumocystis carinii, Paracoccidiodes brasiliensis, Plasmodium falciparum, Plasmodium vivax, Plasmodium ovale, and Plasmodium malariae.

Other examples of antigens include cancer-associated antigens. The terms “cancer-associated antigen” or “tumor-associated antigen” or “tumor-specific marker” or “tumor marker” interchangeably refers to a molecule (typically protein, carbohydrate or lipid) that is preferentially expressed on the surface of a cancer cell in comparison to a normal cell, and which is useful for inducing and/or eliciting an immune response against the cancer cell or tumor. Oftentimes, a cancer-associated antigen is a cell surface molecule that is overexpressed in a cancer cell in comparison to a normal cell, for instance, 2-fold expression, 3-fold expression or more in comparison to a normal cell. Oftentimes, a cancer-associated antigen is a cell surface molecule that is inappropriately synthesized in the cancer cell, for instance, a molecule that contains deletions, additions or mutations in comparison to the molecule expressed on a normal cell. Oftentimes, a cancer-associated antigen will be expressed exclusively on the cell surface of a cancer cell and not synthesized or expressed on the surface of a normal cell. Examples of known TAAs include without limitation, melanoma associated antigens (Melan-A/MART-1, MAGE-1, MAGE-3, TRP-2, melanosomal membrane glycoprotein gp100, gp75 and MUC-1 (mucin-1) associated with melanoma); CEA (carcinoembryonic antigen) which can be associated, e.g., with ovarian, melanoma or colon cancers; folate receptor alpha expressed by ovarian carcinoma; free human chorionic gonadotropin beta (hCGP) subunit expressed by many different tumors, including but not limited to myeloma; HER-2/neu associated with breast cancer; encephalomyelitis antigen HuD associated with small-cell lung cancer; tyrosine hydroxylase associated with neuroblastoma; prostate-specific antigen (PSA) associated with prostate cancer; CA125 associated with ovarian cancer; and the idiotypic determinants of a B cell lymphoma that can generate tumor-specific immunity (attributed to idiotype-specific humoral immune response). Moreover, antigens of human T cell leukemia virus type 1 have been shown to induce specific CTL responses and antitumor immunity against the virus-induced human adult T cell leukemia (ATL) (Haupt, et al, Experimental Biology and Medicine (2002) 227:227-237; Ohashi, et al., Journal of Virology (2000) 74(20):9610-9616).

Other examples of antigens include peptides, proteins, cells or tissues that constitute the molecular targets of an autoimmune response. Said molecular targets are expressed by the tissue(s) or cell(s) targeted by the autoimmune response. Expression of autoimmunity-associated self antigens can be limited to the target tissue or be extended to additional body compartments. Autoimmunity-associated antigens can be initially identified as being targets of autoantibody or T cell immune responses, or based on their selective expression by the target tissue. Some examples of autoimmunity-associated protein antigens are preproinsulin (PPI), glutamic acid decarboxylase (GAD), insulinoma-associated protein 2 (IA-2), islet-specific glucose-6-phosphatase catalytic-subunit-related protein (IGRP), zinc transporter 8 (ZnT8) and chromogranin A for type 1 diabetes; myeloperoxydase and proteinase 3 for granulomatosis with polyangiitis; myelin oligodendrocyte glycoprotein (MOG) and myelin basic protein (MBP) in multiple sclerosis. Examples of autoimmunity-associated peptide antigens are derived from the above said protein antigens following processing by APCs—including DC—and presentation in the context of different HLA Class I or Class II molecules. Therefore, said peptide antigens are different depending not only on their source antigens, but also on the HLA molecules by which they are presented. For example, a list of type 1 diabetes-associated peptide antigens for both mouse and human can be found in DiLorenzo et al., Clin.Exp.Immunol. 148:1, 2007. Autoimmunity-associated peptide antigens also include post-translationally modified aminoacid sequences and sequences derived from alternative splicing isoforms.

In some embodiments, the antigen is a MHC-class I restricted antigen. In some embodiments, the antigen is a HLA-A2 restricted antigen. In some embodiments, the antigen is Melan-A/MART-1 optimized (ELAGIGILTV SEQ ID NO:1) or natural ((EAAGIGILTV (SEQ ID NO:2)) antigen.

Typically step v) is carried out for an amount of time t(c) sufficient to mature DC. Typically, this amount of time t(c) is comprised between about 12 and about 72 hours, preferably between about 16 and about 48 hours, even more preferably for about 24 hours.

Step d) is typically performed by adding fetal calf serum (FCS) or fetal bovine serum (FBS) to the culture medium. Typically, FCS or FBS are added to the medium to reach a final concentration of 10%. FCS and FBS are commercially available from different companies, for example Life Technologies or Sigma. Typically step d) is carried out for an amount of time t(d) sufficient to prime T cells. Typically, this amount of time t(d) is comprised between about 5 days and about 15 days, preferably between about 7 days and about 9 days, even more preferably for about 8 days. In some embodiments, the culture medium is changed every 3 days.

At the end of step d) the primed T cells are isolated for functional analysis. Any functional assay may be used at step e).

In some embodiments, the antigen which has been used for priming the T cells is loaded on MHC Cass I multimers, and the isolated primed T cells are brought into contact with said multimers. HLA multimers assays are well known in the art. To produce multimers, the carboxyl terminus of an MHC molecule, such as, for example, the HLA A2 heavy chain, is associated with a specific peptide epitope, polyepitope or protein (e.g. streptavidin), and treated so as to form a multimeric complex (tetrameric or higher) having bound hereto a suitable reporter molecule, preferably a fluorochrome such as, for example, fluoroscein isothiocyanate (FITC), phycoerythrin, allophycocyanin, Brilliant Violet, Quantum Dot fluorochromes, metallic chemical element such as lanthanides that can be used in CyTOF assays. The multimers produced bind to the distinct set of T cell receptors (TcRs) on a subset of T cells to which the peptide is MHCI restricted. There is no requirement for in vitro T cell activation or expansion. Following binding, and washing of the T cells to remove unbound or non-specifically bound multimers, the number of cells binding specifically to the HLA-peptide multimer may be quantified by standard flow cytometry methods, such as, for example, using BD LSR Fortessa or FACSAria flow cytometers (Becton Dickinson). The multimers can also be attached to paramagnetic particles or magnetic beads to facilitate cell sorting. Such particles are readily available from commercial sources (e.g. Miltenyi). Multimer staining does not kill the labeled cells; therefore cell integrity is maintained for further analysis.

Typically the polyfunctionality of the primed T cells is determined by measuring different parameters which include production, secretion or transport of IFN-gamma, TNF-alpha, IL-2, IL-17A, IL-22, MIP-1beta, Granzyme A, Granzyme B, Perforin, CD107a, IFN-alpha, TGF-beta, G-CSF, GM-CSF, IL-4, IL-5, IL6, IL-7, IL-8, IL-9, IL-10, IL-12, IL-13, IL-17F, IL-18, IL-21, IL-23, IL-28 and IP-10, more particularly IFN-gamma, TNF-alpha, IL-2, IL-17A, IL-17F, IL-22, MIP-1beta, Granzyme A, Granzyme B, Perforin and CD107a. For example, for a single T-cell, preferred possible parameters are Interferon-gamma (IFN-gamma), Tumour Necrosis Factor alpha (TNF-alpha), Interleukin-2 (IL-2), CCL4, also known as Macrophage inflammatory protein-1beta (MIP-1beta), IL-17A, IL-22, Perforin, Granzyme A, Granzyme B and CD107a production.

Any assay well known in the art may be used for measuring the above mentioned parameters. For instance, the parameters of interest are detected with specific antibodies, or with specific oligonucleotides. Such techniques are well known by the man skilled in the art. Typically, data necessary for the above assays are acquired by mass cytometry Time-of-Flight (Newell et al. 2012. immunity. January 27; 36(1):142-52), by chip based single-cell secretomics (Ma et al. 2011. Nat. Med. June; 17(6):738-43) or preferably using a flow cytometer.

In some embodiments, said assay may consist in an enzyme-linked immunospot (ELISpot) assay. Non-adherent cells from pre-culture wells are transferred to a plate which has been coated with the desired anti-cytokine capture antibodies (Abs; e.g., anti-IFN-gamma, -IL-10, -IL-2, -IL-4). Revelation is carried out with biotinylated secondary Abs and standard colorimetric or fluorimetric detection methods such as streptavidin-alkaline phosphatase and NBT-BCIP and the spots counted.

In some embodiments, the method may consist in a cytokine capture assay. This system developed by Miltenyi Biotech is a valid alternative to the ELISpot to visualize antigen-specific T cells according to their cytokine response. In addition, it allows the direct sorting and cloning of the CD8+ T cells of interest.

In some embodiments, the assay may consist in a supernatant cytokine assay. Cytokines released in the culture supernatant are measured by different techniques, such as enzyme-linked immunosorbent assays (ELISA), BD cytometric bead array, Biorad or Millipore cytokine mutiplex assays and others.

In some embodiments, the assay may consist in a CD107 assay. This procedure (Betts et al., J. Immunol. Methods 281:65, 2003) allows the visualization of antigen-specific CD8+ T cells with cytotoxic potential.

In some embodiments, the assay is based on the detection of the upregulation of activation markers (e.g., perforin, granzyme B, CD137). With this procedure, T cell responses are detected by their differential expression of activation markers exposed on the membrane following antigen recognition.

In some embodiments, the assay is a cytotoxic assay that can be performed with any method well known in the art. Briefly, primed T cells according to the present method are put in contact with target cells, and the killing of the targets cells is then evaluated.

In some embodiments, polyfunctionality index that numerically evaluates the degree and variation of polyfuntionality, and enable comparative and correlative parametric and non-parametric statistical tests is calculated according to the teaching of WO2013127904 and Larsen M et al. PLoS One. 2012; 7(7):e42403.

The method of the invention may find various applications, in particular in the field of vaccine.

In some embodiments, the method of the present invention is used for selecting a vaccine for a subject. For example, the state of a patient suffering from HIV infection is not improved despite treatment with a vaccine administered through a particular route of injection at dose A. The use of the method of present invention may evidence a deficiency in the vaccine-induced T cells concerning the simultaneous production of IFN-γ, TNF-α, IL-2 and MIP-1β. Therefore treatment with said vaccine is changed to a treatment with said vaccine administered through another route of injection or at a different dose. Alternatively, said vaccine is modified ex vivo to increase its capacity to induce polyfunctional T cells. An example of said modification is the genetic alteration of the encoded viral antigen or the control elements determining the expression and presentation of said antigen. The method of the present invention may also be used for selecting a vaccine for a subject to induce immune tolerance rather than active immune responses, which is more desirable in the setting of autoimmune diseases. Typically, the vaccine is selected when the desired polyfunctionnality of said vaccine is reached.

In particular, the method of the present invention is particularly suitable for screening adjuvants.

Accordingly, the present invention also relates to a method for screening a plurality of test substances for their adjuvant properties comprising the steps of i) performing the method for testing T cell priming efficacy as above described, wherein the test substance is added at step c), ii) determining the polyfunctionality of the primed T cells as above described, iii) comparing the polyfunctionality determined at step iii) with a predetermined reference polyfunctionality and iv) selecting the test substance as an adjuvant when the polyfunctionality determined at step iii) is superior or equal to the predetermined reference polyfunctionality.

Typically, the predetermined reference polyfunctionality is the polyfunctionality determined for a well-known antigen. As above described, the polyfunctionality may be expressed as an index value for easing the comparing step.

Typically, the test substance is a ligand suitable for the activation of a pathogen recognition receptor as above described.

Typically, the antigen used in the present method is a relevant antigen (i.e. an antigen that will be used in the vaccine composition) or an irrelevant antigen (i.e. an antigen that will not be used in the vaccine composition but is used only for more efficient priming of the T cells (e.g. Melan-A antigen). Accordingly, the screening method of the present invention is thus particularly suitable for identifying specific adjuvants (for specific antigens, or specific for a class of antigens) or identifying universal adjuvants that can be used subsequently for any antigen.

In some embodiments, a combination of test substances is tested.

The screening method as above described may be adapted for determining the more accurate combination between the antigen that will be used in the vaccine composition and the selected adjuvant(s).

Additional studies in animal models may also be performed for further characterizing the screened antigens.

According to another embodiment, the method is used to determine disease prognostics or to predict vaccine responsiveness. For example, the clinical prognosis of a patient suffering from HIV infection is unknown. The use of the method of present invention evidences a deficiency in the antigen-specific T cells of HIV infected patients with negative disease prognostic concerning the simultaneous T cell production of IFN-γ, TNF-α, IL-2 and MIP-1β. Therefore alternative treatments can be initiated, such as Highly Active Antiretroviral Therapy (HAART) and treatments consisting of administration of antibodies blocking negative regulators of T cells, Cytotoxic T-Lymphocyte Antigen 4 (CTLA-4) or Programmed Death Receptor 1 (PD-1).

According to another embodiment, the method is used to determine how a subject is able to develop a specific T cell response (CD4+ and/or CD8+ T cell response(s)) in a physiopathological context. For example the method can thus be particularly suitable for determining whether a subject suffering from a cancer develop a T cell response against the tumor, or will be able to develop an efficient T cell response after being administered with an an anti-cancer vaccine. The method of the invention may also find potential application in the transplantation setting, i.e. to define the potential degree of allo-reactivity to a partially HLA-mismatched graft and thus choose the most efficient immunosuppressive regimens. The method of the invention may also find potential application in the autoimmunity setting, i.e. to define the potential for auto-reactivity to a given self antigen before disease development, which may allow to choose appropriate preventative therapies in at-risk subjects. Typically, the determined polyfunctionality is compared to a predetermined reference polyfunctionality and when the determined polyfunctionality is higher than the predetermined reference polyfunctionality it is meant that the subject will develop a T cell response against the tumor or will achieve a response with the vaccine.

The methods of the present invention, especially the screening method of the present invention, provide a great advantage over the other well-known methods because they can be carried out in a very short time (less than 15 days). Moreover the methods of the present invention offer the advantage to be performed directly on human samples, so that the obtained information is more accurate than the one obtained for example in a non-human sample (e.g. rodent). The method may also be performed in various physiopathological contexts (e.g. cancer, infectious diseases such as AIDS . . . ) and for any category of subject's age (e.g. elderly people). The methods of the present invention could offer the advantage to consume very small amount of samples so that the various paramaters (e.g. adjuvants, combination of adjuvants, antigens, and combinations of antigens . . . ) may be studied.

As exemplified by Examples 3-4, the method of the present invention may be suitable for screening potential adjuvants for vaccination able to influence or boost T cell functional attributes upon priming with specific antigens, and to study the mechanistic basis of these putative effects, directly using human blood samples.

As exemplified by Examples 5-7, the method for testing T cell priming efficacy of the present invention may be suitable for determining whether a subject with an immune deficiency (e.g. HIV infected donors) or a subject with declining immune competence (e.g. elderly people) will be able to develop a protection response after being administered with a vaccine composition.

A further aspect of the present invention relates to a vaccine composition comprising at least one antigen, at least one Ftl3 ligand and at least one TLR8 agonist. In some embodiments, the antigen is a cancer-associated antigen. Typically, the vaccine composition of the present invention is particularly suitable for the treatment of cancer. As used herein, the term “cancer” has its general meaning in the art and includes, but is not limited to, solid tumors and blood borne tumors. The term cancer includes diseases of the skin, tissues, organs, bone, cartilage, blood and vessels. The term “cancer” further encompasses both primary and metastatic cancers. Examples of cancers that may treated by methods and compositions of the invention include, but are not limited to, cancer cells from the bladder, blood, bone, bone marrow, brain, breast, colon, esophagus, gastrointestine, gum, head, kidney, liver, lung, nasopharynx, neck, ovary, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; Leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyo sarcoma; rhabdomyo sarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangio sarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

The invention will be further illustrated by the following figures and examples. However, these examples and figures should not be interpreted in any way as limiting the scope of the present invention.

### FIGURES

FIG. 1. Schematic representation of the naïve CD8+ T cell priming protocol and functional assessment of primed cells.

FIG. 2. In vitro priming of antigen-specific CD8+ T cells from naïve precursors. (A) Representative flow cytometry plots showing ELA/HLA-A*02:01 tetramer staining of donor PBMCs before and after (day 10) priming. Percentages of ELA/HLA-A*02:01 tetramer+ cells within the CD8+ T cell population are indicated. (B) Representative flow cytometry plots showing the phenotypes of total, naïve and memory purified CD8+ T cells used for in vitro priming. Percentages of naïve CD8+ T cells (CD45RA+ CCR7+) are indicated. (C) Tetramer staining of ELA-specific CD8+ T cells at day 10 post-priming is shown for each of the starting populations depicted in (B). Purified naïve and memory CD8+ T cell populations were supplemented separately with autologous CD8-depleted PBMCs to initiate priming. Percentages of ELA/HLA-A*02:01 tetramer+ cells within the CD8+ T cell population are indicated. Data shown are representative of three independent experiments. E. Expansion kinetics of ELA/HLA-A*02:01 tetramer+ CD8+ T cells after antigen-specific priming of PBMCs from 10 different healthy donors.

FIG. 3. Memory differentiation phenotype of primed CD8+ T cells. (A) Representative phenotype of ELA/HLA-A*02:01 tetramer+ (black) or total (grey) CD8+ T cells at day 0 and at day 10 post-priming. Percentages of ELA/HLA-A*02:01 tetramer+ naïve CD8+ T cells (CD45RA+ CCR7+) are shown. (B) Proportions of central memory like (CD45RA− CCR7+) or effector memory like (CD45RACCR7-) cells within expanded ELA tetramer+ CD8+ T cells at day 10 post priming using GM-CSF/IL-4 or FLT3L. Bars indicate the median.

FIG. 4. Magnitude and kinetics of GM-CSF/IL-4 or FLT3L primed CD8+ T cells. (A) Magnitude of ELA tetramer+ CD8+ T cells 10 days after initiation of priming using GMCSF/IL-4 or FLT3L on PBMCs of healthy middle aged adults. Bars indicate the median. (B) Expansion kinetics of ELA tetramer+ CD8+ T cells upon priming using GM-CSF/IL-4 or FLT3L on PBMCs of healthy middle aged adults.

FIG. 5. Higher polyfunctionality of antigen specific CD8+ T cells primed using FLT3L. (A) Representative example of simultaneous multifunctional assessment of ELA tetramer+ CD8+ T cells after initiation of priming using GM-CSF/IL-4 or FLT3L. Degranulation (CD107a) and cytokine/chemokine secretion (IFN-γ, TNF-α, MIP-1β and IL-2) by ELA tetramer+ CD8+ T cells upon restimulation with cognate peptide for 6 hours 10 days after priming using GM-CSF/IL-4 or FLT3L was assessed by flow cytometry. Percentages of cells in the different quadrants are shown. Plots are gated on CD8+ tetramer+ cells. (B) The pie charts depict the background adjusted multifunctional behaviour (one to five functions: CD107a, IFN-γ, TNF-α, IL-2 and MIP-1β) of ELA specific CD8+ T-cell populations (n=14) primed using GM-CSF/IL-4 or FLT3L. For simplicity, responses are grouped by number of functions. (C. Polyfunctionality index values of ELA specific CD8+ T cells at day 10 post priming using GM-CSF/IL-4 or FLT3L. The P-value monitoring differences between GM-CSF/IL-4 or FLT3L was calculated using a non-parametric Mann-Whitney test. Bars indicate the median.

FIG. 6. Impact of TLR4L or TLR8L on the priming of antigen specific CD8+ T cells in vitro. Magnitude of ELA tetramer+ CD8+ T cells 10 days after initiation of priming using combinations of GM-CSF/IL-4 or FLT3L with the standard cytokine cocktail (i.e. TNF-α+IL-1β+IL-7+PGE2), TLR4L or TLR8L. Bars indicate the median. * indicates a P value below 0.05.

FIG. 7. Influence of TLR4L or TLR8L on the cytotoxic potential of in vitro primed CD8+ T cells. (A) Percentage of ELA tetramer+ CD8+ T cells (primed using combinations of GM-CSF/IL-4 or FLT3L with the standard cytokine cocktail (i.e. TNF-α+IL-1β+IL-7+PGE2), TLR4L or TLR8L) that express the cytotoxin perforin. (B) Percentage of ELA tetramer+ CD8+ T cells (primed using combinations of GM-CSF/IL-4 or FLT3L with the standard cytokine cocktail (i.e. TNF-α+IL-1β+IL-7+PGE2), TLR4L or TLR8L) that express the cytotoxin Granzyme B. Bars indicate the median. The P-values monitoring differences between priming conditions were calculated using a nonparametric Mann-Whitney test. *, ** and *** indicate P values below 0.05, 0.01, 0.001 respectively.

FIG. 8. Higher polyfunctionality of antigen specific CD8+ T cells primed using FLT3L and TLR8L (A) The pie charts depict the background adjusted multifunctional behaviour (one to five functions: CD107a, IFN-γ, TNF-α, IL-2 and MIP-1β) of ELA-specific CD8+ T-cell populations (n=14) primed using GM-CSF/IL-4 or FLT3L, combined with the standard cytokine cocktail (i.e. TNF-α+IL-1β+IL-7+PGE2), TLR4L or TLR8L. For simplicity, responses are grouped by number of functions. (B) Polyfunctionality index values of ELA specific CD8+ T cells at day 10 post priming using GM-CSF/IL-4 or FLT3L, combined with the standard cytokine cocktail (i.e. TNF-α+IL-1β+IL-7+PGE2), TLR4L or TLR8L. The P-values monitoring differences between priming conditions were calculated using a nonparametric Mann-Whitney test. Bars indicate the median. * and * * * indicate P values below 0.05 and 0.001 respectively.

FIG. 9. Higher antigen sensitivity of antigen specific CD8+ T cells primed using FLT3L and TLR8L. (A) Antigen sensitivity of ELA tetramer+ CD8+ T cells primed using combinations of GM-CSF/IL-4 or FLT3L with the standard cytokine cocktail (i.e. TNF-α+IL-1β+IL-7+PGE2), TLR4L or TLR8L. Antigen sensitivity is defined as the concentration required to achieve half-maximal activity (EC50) in peptide titration assays of (normalized) IFN-γ secretion by ELA tetramer+ CD8+ T cells. This is representative of three experiments. (B) Recognition of melanoma tumor cell lines by ELA tetramer+ CD8+ T cells primed using combinations of GM-CSF/IL-4 or FLT3L with the standard cytokine cocktail, TLR4L or TLR8L. the polyfunctional profile (one to five simultaneous functions: CD107a, IFN-γ, TNF-α, IL-2 and MIP-1β) of ELA tetramer+ CD8+ T cells is depicted using pie charts for simplicity.

FIG. 10. Impact of TLR8L on T-bet expression in in vitro primed CD8+ T cells. Percentage of ELA tetramer+ CD8+ T cells (primed using combinations of GM-CSF/IL-4 or FLT3L with the standard cytokine cocktail, TLR4L or TLR8L) that express the transcription factor T-bet. * and *** indicate P values below 0.05 and 0.001 respectively.

FIG. 11. Blocking IL-12 reduces TRL8L mediated benefit on primed T cell functionality. (A) T-bet, Granzyme B and Perforin expression within ELA tetramer+ CD8+ T cells primed using FLT3L and the standard cytokine cocktail or TLR8L in the presence or in the absence of anti-IL12 blocking antibodies. (B) Polyfunctional profile (one to five simultaneous functions: CD107a, IFN-γ, TNF-α, IL-2 and MIP-1β) of ELA tetramer+ CD8+ T cells (primed using FLT3L and the standard cytokine cocktail or TLR8L) in the presence or in the absence of anti-IL12 blocking antibodies.

FIG. 12. Response kinetics and cytotoxic potential of CD8⁺ T-cells primed in vitro. (A) Representative flow cytometry data showing the kinetics of ELA-specific CD8⁺ T-cell priming in the presence of GM-CSF/IL-4 and a cocktail of inflammatory cytokines Plots are gated on viable CD3⁺ lymphocytes after aggregate exclusion. Numbers refer to percentages of tetramer⁺ cells within the total CD8⁺ population. (B) Response magnitudes for ELA-specific CD8⁺ T-cell populations primed under different conditions. Antigen only (no maturation signal), cytokine cocktail (TNF, IL-1β, PGE2 and IL-7), TLR4L (LPS) or TLR8L (ssRNA40) were each used in combination with either GM-CSF/IL-4 or FLT3L supplementation. (C) Representative flow cytometry plots showing granzyme B (top panel) or perforin (bottom panel) expression by ELA-specific CD8⁺ T-cells primed under different conditions. (D) Granzyme B (top graph) and perforin (bottom graph) expression by ELA-specific CD8⁺ T-cells primed under different conditions. (E) Representative flow cytometry data from a FATAL cytotoxicity assay showing the disappearance of ELA peptide-pulsed PBSE⁺ HLA-A2⁺ B-LCL target cells relative to non-peptide-pulsed PBSE⁻ HLA-A2⁺ B-LCL control cells in the presence of ELA-specific CD8⁺ T-cells primed under different conditions. Numbers indicate the percentages of control (upper left) and target (upper right) B-LCL cells. (F) Specific lysis of HLA-A2⁺ B-LCL target cells presenting the indicated concentrations of exogenously-loaded ELA peptide in the presence of ELA-specific CD8⁺ T-cells primed under different conditions. A non-cognate population of CD8⁺ T-cells derived from the same HLA-A2⁺ donor and cultured under similar conditions to the ELA-specific CD8⁺ T-cell population was used as a control. Error bars represent SD from the mean of two replicates. In (B) and (D), horizontal bars indicate median values. Statistical comparisons between groups were performed using the Wilcoxon signed rank test; *P<0.05, **P<0.01, ***P<0.001.

FIG. 13. Polyfunctionality and antigen sensitivity of CD8⁺ T-cells primed in vitro. (A) Representative flow cytometry plots showing cytokine production (IFNγ, MIP-1β, TNF and IL-2) and degranulation (CD107a) in response to antigen stimulation. ELA-specific CD8⁺ T-cells primed under the indicated conditions were incubated with media alone (top row) or ELA peptide-pulsed HLA-A2⁺ B-LCL target cells (middle and bottom rows). Primed ELA-specific CD8⁺ T-cells were quantified as tetramer⁺ cells within the total CD8⁺ population (left column). Function plots are gated on viable CD3⁺ CD8⁺ tetramer⁺ lymphocytes (middle and right columns). Numbers in each quadrant refer to the percentages of primed ELA-specific CD8⁺ T-cells expressing the indicated combinations of functions. (B) Averaged pie chart representations of background-adjusted polyfunctional profiles for ELA-specific CD8⁺ T-cells primed under different conditions from 10 healthy donors. Pie segments and colours correspond to the proportions of ELA-specific CD8⁺ T-cells expressing the indicated number of functions, respectively. (C) Polyfunctionality indices for ELA-specific CD8⁺ T-cell populations, calculated from the data depicted in (B). Horizontal bars indicate median values. Statistical comparisons between groups were performed using the Wilcoxon signed rank test; *P<0.05, ***P<0.001. (D) Normalized IFNγ production curves for ELA-specific CD8⁺ T-cells primed under different conditions. HLA-A2⁺ B-LCL target cells were pulsed with ELA peptide across a range of concentrations and used to stimulate ELA-specific CD8⁺ T-cells in standard intracellular cytokine staining assays. (E) Polyfunctional profiles of ELA-specific CD8⁺ T-cells responding to natural levels of the Melan-A/MART-1 antigen presented by an HLA-A2⁺ melanoma cell line. An HLA-A2⁺ Melan-A/MART-1⁻ tumor cell line was used as a negative control (data not shown). In (D) and (E), data represent two independent experiments conducted with HLA-A2⁺ PBMCs from two different donors.

FIG. 14. T-bet and IL-12 levels determine the functional quality of FLT3L/TLR8L-primed CD8⁺ T-cells. (A) Representative flow cytometry plots showing intracellular T-bet expression (white histograms) by ELA-specific CD8⁺ T-cells primed under different conditions. (B) Intracellular T-bet expression by ELA-specific CD8⁺ T-cells primed under different conditions. (C) Representative flow cytometry plots showing intracellular T-bet expression (white histograms) by ELA-specific CD8⁺ T-cells primed with FLT3L plus the indicated maturation signals in the presence or absence of an α-IL-12p70 blocking antibody. (D) Intracellular T-bet expression by ELA-specific CD8⁺ T-cells primed as in (C). (E) Granzyme B (top graph) and perforin (bottom graph) expression by ELA-specific CD8⁺ T-cells primed as in (C). Error bars indicate SD from the mean. (F) Polyfunctional profiles of ELA-specific CD8⁺ T-cells primed as in (C). Data are averaged over two independent experiments with ELA peptide-pulsed HLA-A2⁺ B-LCL target cells. In (A) and (C), grey histograms depict isotype control staining and vertical dotted lines indicate the mean fluorescence intensity (MFI) of T-bet staining for the weakest priming condition. In (B) and (D), horizontal bars indicate median values. Statistical comparisons between groups were performed using the Wilcoxon signed rank test; *P<0.05, **P<0.01, ***P<0.001.

FIG. 15. Reduced in vitro CD8+ T cell priming efficacy in elderly donors. (A) Frequencies of ELA/HLA-A*02:01 tetramer+ CD8+ T cells after in vitro priming (day 10) in middle aged healthy (between 25 and 55 years old) adults (n=20, Mid) and elderly (>70 years old) adults (n=46, Old). (B) Phenotypic distribution (based on CD45RA and CCR7 expression) of ELA/HLA-A*02:01 tetramer+ CD8+ T cells at day 10 post-priming in healthy middle-aged or elderly adults. (C) Representative flow cytometry plots showing standard or CD8-null tetramer staining to identify total or high avidity ELA-specific CD8+ T cells, respectively. (D) Frequencies of high avidity ELA-specific CD8+ T cells in middle-aged healthy adults (n=17) and elderly adults (n=19) with strong expansions (>0.4%) of total ELA/HLA-A*02:01 tetramer+ CD8+ T cells after in vitro priming. (E) CD8-null/standard ratios for ELA/HLA-A*0201 tetramer+ CD8+ T cells in middle-aged healthy adults (n=17) and elderly adults (n=19) after in vitro priming. Bars indicate median values. Statistical analyses were conducted using the Mann-Whitney U-test.

FIG. 16. Reduced in vitro CD8+ T cell priming efficacy in HIV-1 infected patients. (A) Frequencies of ELA/HLA-A*02:01 tetramer+ CD8+ T cells after in vitro priming (day 10) in healthy controls (n=20, Ctl) and HIV-1 infected adults (n=46, HIV). (B) Frequencies of high avidity ELA-specific CD8+ T cells in healthy controls (n=17, Ctl) and HIV-1 infected adults (n=22, HIV) with strong expansions (>0.4%) of total ELA/HLA-A*02:01 tetramer+ CD8+ T cells after in vitro priming. (C) CD8-null/standard ratios for ELA/HLA-A*0201 tetramer+ CD8+ T cells in middle-aged healthy adults (n=17) and HIV-1 infected adults (n=22) after in vitro priming. Bars indicate median values. Statistical analyses were conducted using the Mann-Whitney U-test. (D) Frequencies of ELA/HLA-A*02:01 tetramer+ CD8+ T cells after in vitro priming (day 10) in different group of HIV-1 infected donors divided into old treated patients (>65 years old; Old Tx), middle aged patients (between 25 and 55 years old; Mid Tx), and untreated elite controllers (EC).

FIG. 17. CD8+ T cell priming capacity and size of the naïve T cell compartment. (A) Representative flow cytometry plots showing ELA-specific CD8+ T cell precursors in a healthy donor before and after enrichment from 10⁸ PBMCs. Percentages of ELA/HLA-A*02:01 tetramer+ cells within the CD8+ T cell population are indicated. (B) Correlation between ELA-specific CD8+ T cell precursor (CD45RA+ CCR7+) frequency and ELA/HLA-A*02:01 tetramer+ CD8+ T cell frequency after in vitro priming in middle-aged healthy adults (n=20). (C) Correlation between naïve CD8+ T cell frequency and ELA/HLA-A*02:01 tetramer+ CD8+ T cell frequency after in vitro priming in elderly (>70 years old) adults. (D) Correlation between naïve CD8+ T cell frequency and ELA/HLA-A*02:01 tetramer+ CD8+ T cell frequency after in vitro priming in HIV-1 infected donors. Correlations were determined using the Spearman's rank test

FIG. 18. In vitro assessment of CD8+ T cell priming efficacy in TBE vaccinated elderly donors. (A) Binding (left panel) and neutralizing (right panel) antibody titers specific for TBEv in elderly (>70 years old) adults before and at weeks 8 and 28 after the first immunization. Top and bottom quartiles of titer values at weeks 8 or 28 were used to define good (n=12) and poor (n=12) TBE vaccine responders respectively. (B) Frequencies of ELA/HLA-A*02:01 tetramer+ CD8+ T cells after in vitro priming in good or poor TBE vaccine responders. Bars indicate median values. The statistical comparison was conducted using the Mann-Whitney U-test. (C) Association between in vitro CD8+ T cell priming efficacy prior to TBE vaccination and TBE vaccine responsiveness based on anti-TBEv antibody titers. Statistical significance was assessed using the χ² test. (D) Correlation between in vitro CD8+ T cell priming efficacy prior to TBE vaccination and the de novo TBE-specific T cell responses determined by IFN-γ ELISpot at week 26 post-immunization. The correlation was determined using the Spearman's rank test.

### EXAMPLES

### Example 1

### Material & Methods

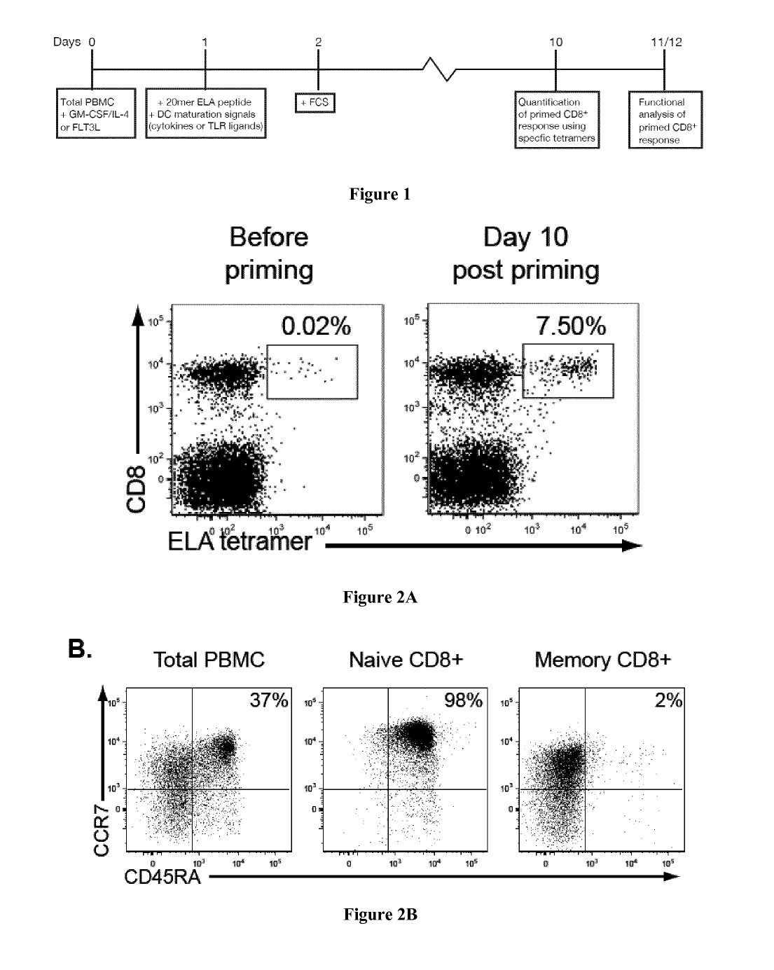

In vitro priming of antigen-specific CD8+ T cell precursors (FIG. 1). Naïve precursors specific for the HLA-A*02:01-restricted epitope ELAGIGILTV (ELA) (SEQ ID NO:1) derived from Melan-A/MART-1 (residues 26-35) were primed in vitro from HLA-A*02:01 blood donors. Thawed PBMCs were resuspended in AIM medium (Invitrogen), plated out at 2.5×106 cells/well in a 48-well tissue culture plate and stimulated with the 20mer peptide YTAAEELAGIGILTVILGVL (SEQ ID NO:2), which contains the optimal epitope in heteroclitic form, at a concentration of 1 μM in the presence of FLT3 ligand (50 ng/mL) or a combination of GM-CSF (0.2 μg/ml) and IL-4 (50 ng/ml) (R&D Systems). After 24 hours (day 1), maturation was induced by the addition of a standard cytokine cocktail comprising TNF-α (1000 U/mL), IL-1β (10 ng/mL), IL-7 (0.5 ng/mL) and PGE2 (1 μM) (R&D Systems), or TLR4 ligand (0.1 μLg/ml) or TLR8 ligand (0.5 μg/ml) (Invivogen). On day 2, fetal calf serum (FCS; Gibco) was added to reach 10% by volume per well; fresh RPMI-1640 (Gibco) enriched with 10% FCS was subsequently used to replace the medium every 3 days. The frequency, phenotype and functional profiling of ELA-specific CD8+ T cells were typically determined on day 10 or 11. Purified naïve and memory CD8+ T cell subsets for priming experiments were obtained using T Cell Enrichment Column Kits (R&D Systems).

Study subjects and samples. Three groups of volunteers were enrolled for this work: (i) middle-aged healthy adults (median age, 35 years); (ii) elderly (>70 years old) healthy adults (median age, 78 years); and, (iii) HIV-1-infected patients with undetectable viral loads (median age, 43 years). Individuals undergoing immunosuppressive therapy were excluded from the study. Among the elderly, we studied an additional cohort of 40 donors who received a full tick-borne encephalitis (TBEV) vaccination course of three injections at week 0, 4 and 24 with a licensed inactivated whole virus vaccine (FSME Immun®, Baxter) as part of a clinical trial (NCT00461695). All individuals were >70 years, healthy (no chronic diseases, ≦one medication) and TBEV-naïve and -seronegative. Immune response assays were conducted prior to vaccination, and at weeks 8 and 28 post-vaccination for humoral and week 26 for cellular immune responses. The HIV-1-infected patients were divided into three subgroups: (i) treatment-naïve patients with effective natural control of viral replication for at least 5 years (elite controllers); (ii) non-elderly (<65 years old) patients (median age, 44 years) on continuous ART for at least 3 years (non-elderly treated); and, (iii) elderly (>65 years old) patients (median age, 69 years) on continuous ART for at least 3 years (elderly treated). All participants provided written informed consent. The study was approved by the local institutional ethics committee (i.e. Comité de Protection des Personnes of the Pitié Salpétrière Hospital, Paris and cantonal ethics committee, Zürich, Switzerland). Venous blood samples were drawn into anti-coagulant tubes and PBMCs were isolated by density gradient centrifugation according to standard protocols.

Flow cytometry reagents. Fluorochrome-conjugated ELA/HLA-A*A2:01 tetramers were produced and used as described previously (Price et al. 2005. J Exp Med 202, 1349-61). The D227K/T228A compound mutation was introduced into the a 3 domain of HLA-A*A2:01 to generate CD8-null tetramers, which enable the selective identification of high avidity antigen-specific CD8+ T cells (Purbhoo et al. 2001. J Biol Chem 276, 32786-92; and Wooldridge et al. 2009. Immunology 126, 147-64). The following directly conjugated monoclonal antibodies were used according to standard protocols: anti-CD8 APC-Cy7 (Caltag), anti-CD27 Alexa700 (BioLegend), anti-CD45RA ECD, anti-Granzyme B-PE-TexasRed (Beckman Coulter), anti-CCR7 PE-Cy7, anti-CD107a-PE-Cy5, anti-IL2-APC, anti-IFNγ-Alexa700, anti-TNF-PE-Cy7, anti-perforin-FITC, anti-Tbet-FITC (BD Biosciences), anti-MIP-1β-FITC, anti-IL12-flurochrome? (R&D Systems). Samples were acquired using a Fortessa flow cytometer (BD Biosciences). Data analysis was conducted with FACSDiva 7.0 (BD Biosciences) and FlowJo v9 (TreeStar Inc.) software. Ex vivo frequencies of ELA-specific precursors were determined from pre-enriched PBMC samples according to published procedures (Alanio et al. 2010. Blood 115, 3718-25; and Iglesias et al. 2013. Immunol Lett 149, 119-22).

Polyfunctionality analysis. In vitro expanded CD8⁺ T-cells and HLA-A2⁺ LCLs pulsed with ELA 10mer peptide (at 1 μM) were incubated for 1 hour with anti-CD 107a and a further 5 hours in the presence of monensin (2.5 μg/mL; Sigma-Aldrich) and brefeldin A (5 μg/mL; Sigma-Aldrich) at 37° C./5% CO₂. Negative controls were processed likewise in the absence of peptide. Staining for intracellular markers (i.e. IL2 IFNγ TNFα and MIP1β) was performed as described previously (Almeida et al. 2007. J Exp Med 204, 2473-85). Data were acquired using a Fortessa flow cytometer (BD Biosciences) and analyzed with FlowJo software version 9.4.4 (TreeStar Inc.). Pie plots were constructed using spice software and polyfunctionality indices were calculated as described previously (Larsen et al. 2012. PLoS One 7, e42403).

Analysis of TBEV-specific humoral and cellular immune responses. TBEv specific antibody titers were measured before (week 0) during (week 8) and after (week 28) the TBE vaccination course by ELISA and TBEv-neutralization assay according to published protocols (Stiasny et al. 2012. J Clin Viol 54, 115-20). The TBEv specific cellular immune response was assessed at week 0 and 26 by IFNγ enzyme-linked immunosorbent spot assay (ELISpot) using pools of overlapping peptides for all structural proteins of TBEv. Briefly, 2×10⁵ thawed PBMCs/well from week 0 and week 26 of the same donor were stimulated in anti-IFNγ (clone 1-D1K, Mabtech) coated 96-well ELISpot-plates (MAIP S45, Millipore) for 18 h with 2×10⁴ freshly generated autologous monocyte derived DCs. For antigen-specific stimulation, five pools of overlapping peptides encompassing all structural proteins of TBEv were used at 1 μg/ml final peptide concentration (15-mers overlapping by 5; BMC Microcollections, Germany). Washed plates were incubated with anti-IFNγ-Biotin (7-B6-1, Mabtech) followed by Streptavidin-alkaline Phosphatase (Mabtech), developed with color reagents (170-6432, Biorad) and analyzed in an automated ELISpot reader (AID). The number of total spot forming units (SFU) was calculated after background subtraction of the unstimulated control.

Statistical analysis. Univariate statistical analyses were performed using GraphPad Prism software. Groups were compared using the non-parametric Mann-Whitney or χ2 tests. Spearman's rank test was used to determine correlations. P values below 0.05 were considered significant.

### Example 2

### In Vitro Model of Antigen-Specific Naïve CD8⁺ T Cell Priming

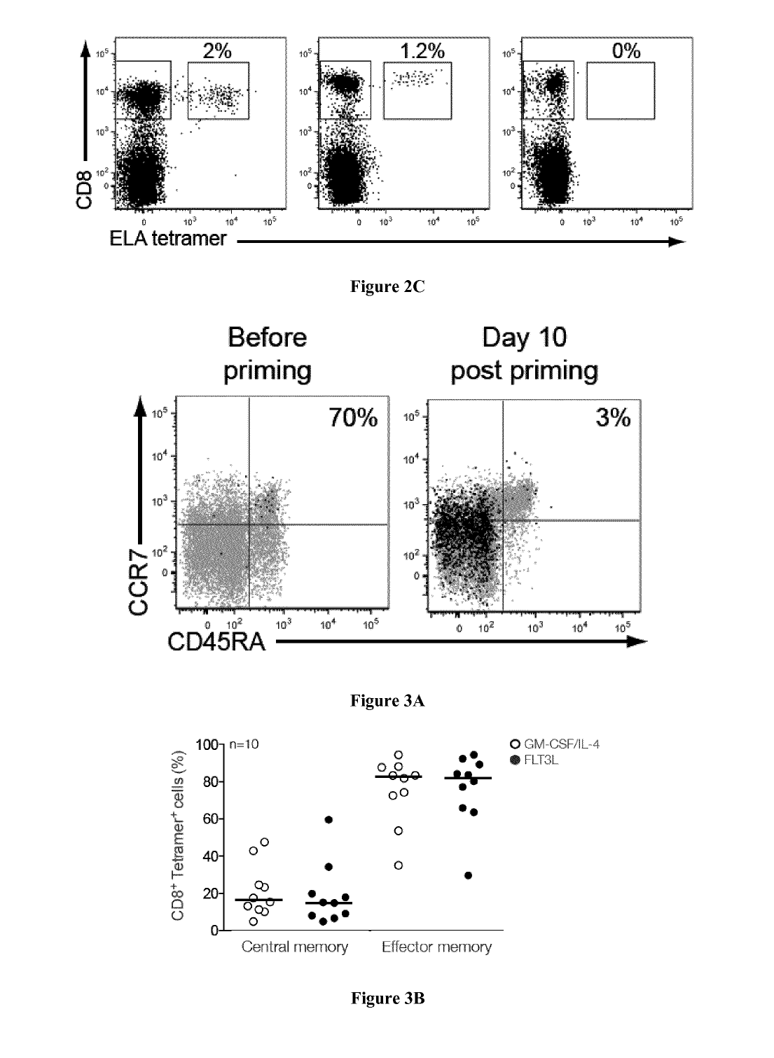

The frequency of circulating antigen-reactive CD8⁺ T cell precursors in humans is typically very low, often in the order of 1 cell per million within the lineage as a whole (Alanio et al. 2010. Blood 115, 3718-25; and Iglesias et al. 2013. Immunol Lett 149, 119-22). To circumvent this biological obstacle to the reliable study of antigen-specific priming in vitro, we developed an assay based on the expansion of naïve CD8⁺ T cells with reactivity against the HLA-A2-restricted Melan-A/MART-1 epitope ELAGIGILTV (ELA from hereon), which occur in individuals with the appropriate allotype at frequencies between 10 and 100 precursors per million CD8⁺ T cells (Dutoit et al. 2002. J Exp Med 196, 207-16; and Zippelius et al. 2002. J Exp Med 195, 485-94). This approach enabled reproducible in vitro priming from a small number of PBMCs (2.5×10⁶ in our assays) in response to stimulation with the cognate ELA epitope encompassed within a longer (i.e. 20-mer) synthetic peptide. To boost antigenpresenting cells and optimize T cell priming, the stimulation cocktail incorporated FLT-3 ligand, TNF-α, IL-1β, PGE2 and IL-7 (Martinuzzi et al. 2011. Blood 118, 2128-37). ELA-specific CD8⁺ T cells were quantified by flow cytometry using fluorochrome-labeled ELA/HLA-A*02:01 tetramers (FIG. 2A). Although it is established that ELA reactive CD8⁺ T cells in healthy donors can be defined as naïve T cells (characterized by a CD45RA⁺ CCR7⁺ phenotype, a high TREC content and long telomeres) (Dutoit et al. 2002. J Exp Med 196, 207-16; and Zippelius et al. 2002. J Exp Med 195, 485-94), we checked that ELA specific T cell priming in these donors was indeed occurring within the naïve (and not memory) CD8⁺ T cell compartment. For this purpose, we mixed purified naïve or memory CD8⁺ T cells separately with autologous CD8-depleted PBMCs to initiate priming (FIG. 2B). Antigen-specific expansion was only observed with naïve CD8⁺ T cells (FIG. 2C), thereby validating the experimental system.

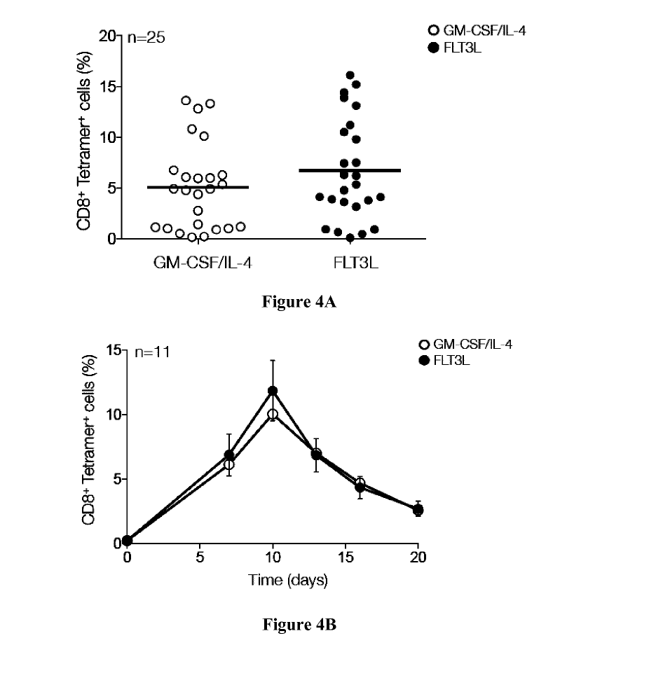

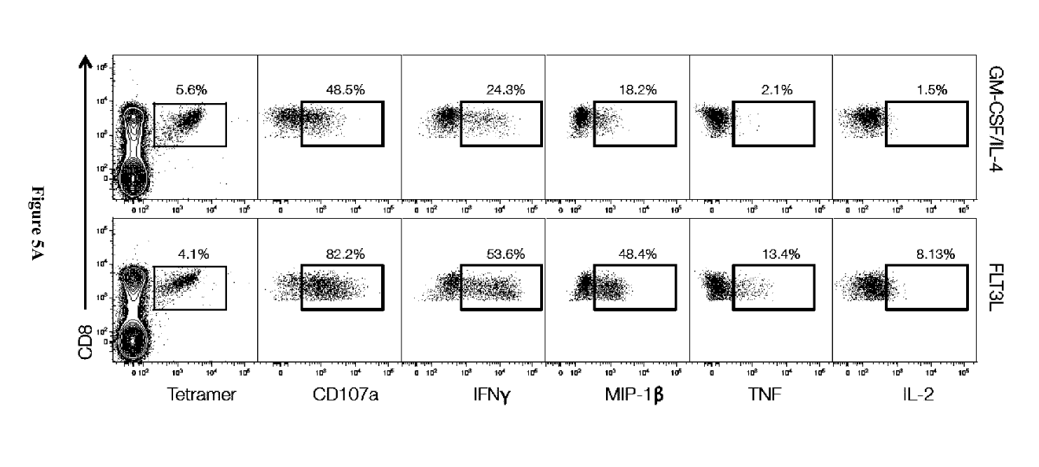

The differentiation phenotype of ELA-specific CD8⁺ T cells expanded upon priming was assessed according to CD45RA and CCR7 surface expression. After expansion, the majority of ELA-specific CD8⁺ T cells present a memory phenotype (CD45RA⁻ CCR7⁻) (FIG. 3A), reflecting the differentiation and expansion of ELA-reactive naïve CD8⁺ T cell precursors (CD45RA⁺ CCR7⁺) upon priming. There was no difference in the differentiation phenotype of expanded ELA-specific CD8⁺ T cells comparing priming using GM-CSF/IL-4 or FLT3L (FIG. 3B). We observed no significant differences between GM-CSF/IL-4 or FLT3L either with regards to the magnitude of expanded ELA-specific CD8⁺ T cells 10 days after priming (FIG. 4A). Optimization experiments with healthy donor PBMC samples revealed maximal ELA-specific CD8⁺ T cell expansion at 10-11 days post-priming (FIG. 4B). This time course was adopted in all subsequent assays.

### Example 3

### Influence of Potential Adjuvants on the Quality of Antigen-Specific CD8⁺ T Cells Primed In Vitro

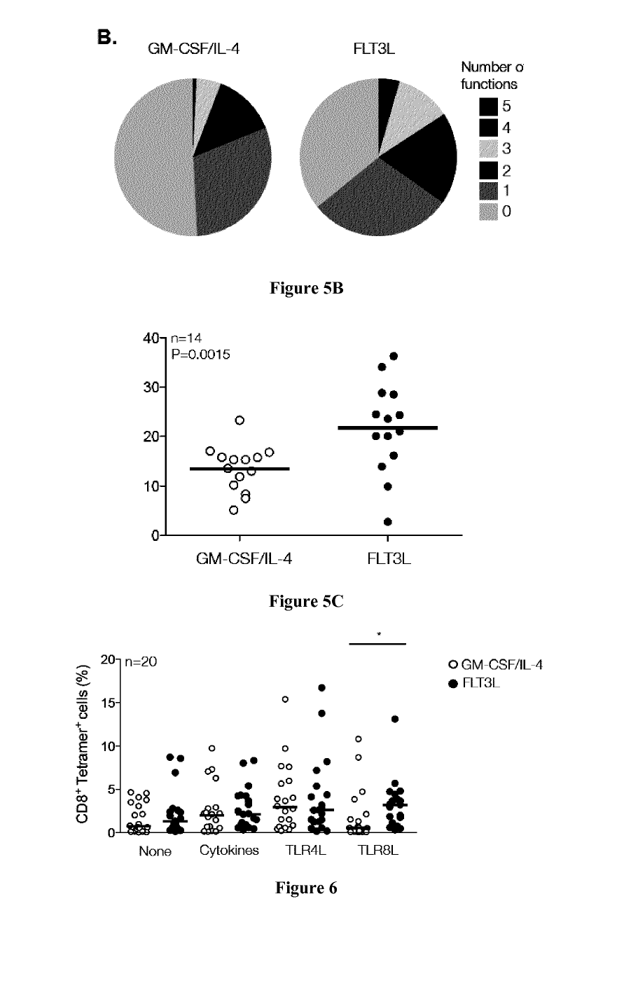

Increasing evidence suggest that the quality, rather than the quantity, of T cell responses is key for their efficacy against viruses or tumors (Appay et al. 2008. Nat Med 14, 623-8). It is therefore important to be able to induce high quality T cells using vaccines in humans. A functional attribute that is regularly measured to assess the quality of T cells is their polyfunctional profile. This is the capacity of a cell to show different functions (i.e. effector cytokine production, cytotoxic potential) simultaneously upon stimulation with its cognate antigen. We therefore assessed the polyfunctional profile of ELA-specific CD8⁺ T cells expanded using our protocol and compare the attributes between priming using GM-CSF/IL-4 or FLT3L (FIG. 5A). We found that FLT3L primed CD8⁺ T cells displayed a significantly more robust polyfunctional profile than GM-CSF/IL-4 cells (FIGS. 5B and 5C).

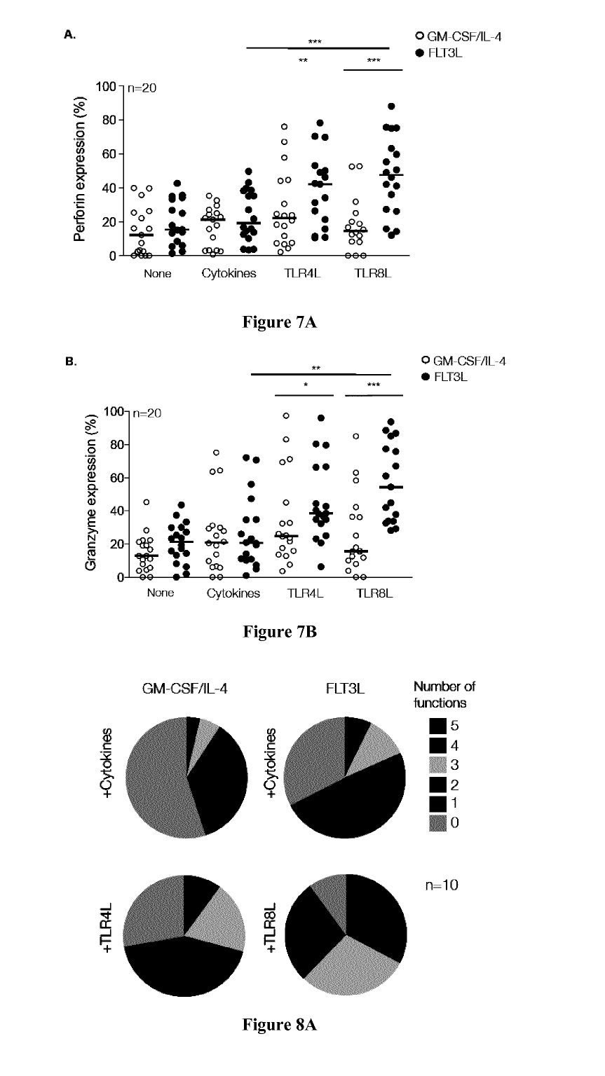

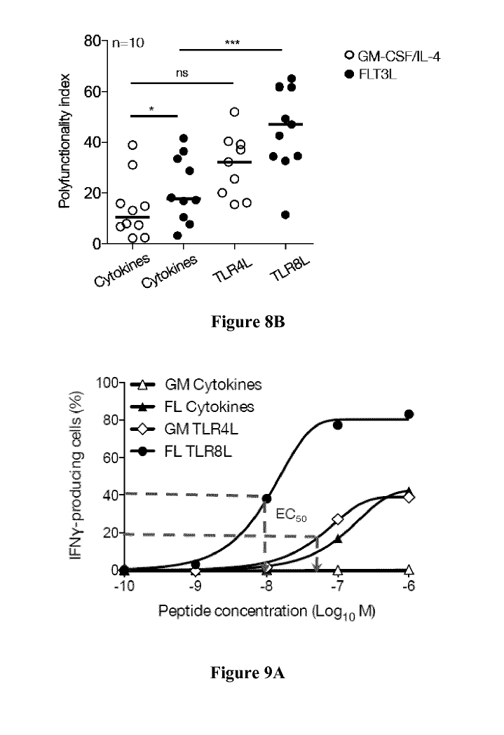

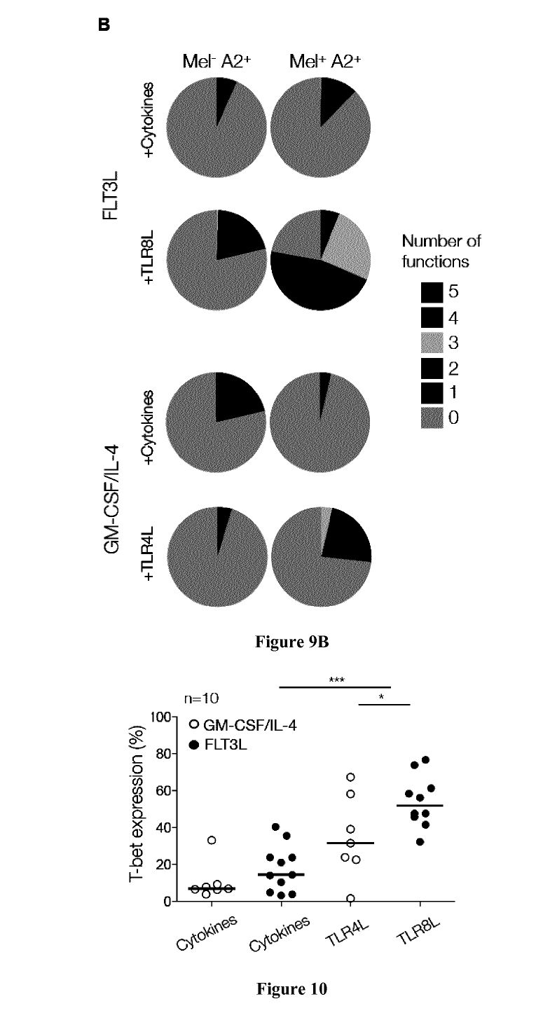

We next investigated the potential effect of TLR ligands on the CD8⁺ T priming using our approach. In particular, we concentrated our study on TLR8L, whose potential as an adjuvant to boost T cell responses has been poorly studied. We observed only marginal effects on the expansion of ELA-specific CD8⁺ T cells by substituting the DC maturing cytokine cocktail by TLR8L or the classically used TLR4L (FIG. 6). However, intracellular expression of the cytotoxins perforin and granzyme B (which are key factors for T cell cytotoxicity) appeared to be particularly enhanced in CD8 T cells primed using TLR8L, in particular in combination with FLT3L (FIG. 7). On the same line, the polyfunctional profile of ELA-specific CD8⁺ T cells expanded using a combination of FLT3L and TLR8L was particularly robust (FIG. 8). Of note, the antigen sensitivity or functional avidity of FLT3L+TLR8L primed CD8⁺ T cells was higher than in the one of CD8⁺ T cells primed in other conditions (FIG. 9A), so that FLT3L+TLR8L primed CD8⁺ T cells were the only cells able to display a functional response against a tumor cell line expressing naturally the Melan-A antigen on its surface (FIG. 9B). Overall, these data show that the use of TLR8L together with FLT3L represents a potent combination to induce antigen specific CD8⁺ T cells with superior qualitative attributes. TLR8L may thus present a particular interest for use as adjuvant in vaccines aimed at inducing effective CD8⁺ T cell responses.

### Example 4

### Mechanistic Understanding of Superior Quality of FLT3L+TLR8 Induced CD8⁺ T Cells

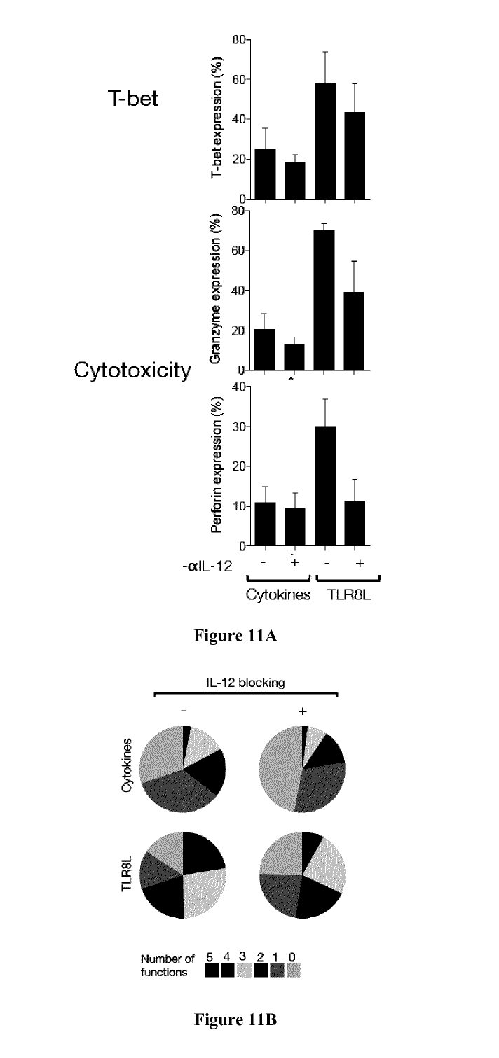

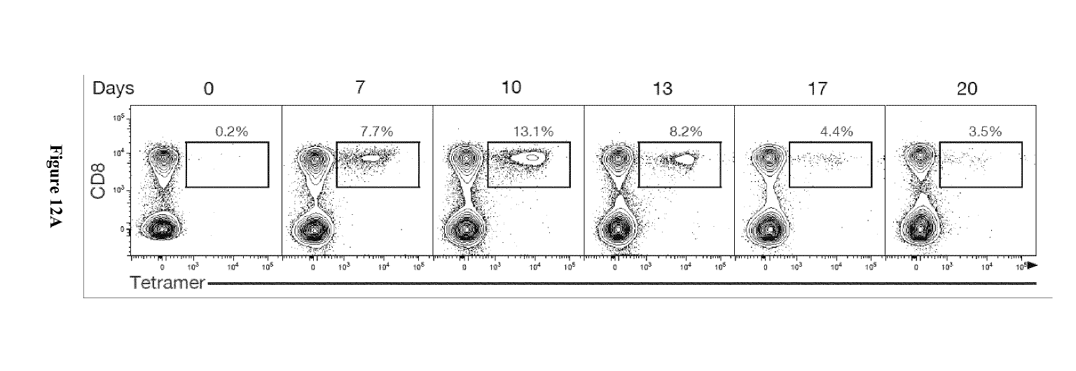

Our in vitro system also enables us to investigated putative mechanisms underlying the induction of highly functional antigen specific CD8+ T cells using FLT3L+TLR8L priming. The production of effector cytokines and cytotoxins can be orchestrated at the gene expression level and is regulated by the expression of Tbet, or Tbx21 protein, a Th1 cell-specific transcription factor. We observed that FLT3L+TLR8L priming resulted in a significantly higher Tbet expression within expanded ELA-specific CD8⁺ T cells (FIG. 10), thus explaining the robust polyfunctional profile of these cells. In addition to signaling via the TCR, through specific interaction with the peptide MHC complex, Tbet expression is induced via the IL12/IL12R signaling pathway. IL12 is an important Th1 cytokine usually secreted by mature dendritic cells to drive T cell differentiation and function. We used anti-IL12 blocking antibodies in our priming assay to investigate the potential role of IL12 on the effective T cell priming by FLT3L+TLR8L. Blocking IL12 during priming resulted in decreasing perforin and granzyme B intracellular expression as well as polyfunctionality of expanded ELA-specific CD8⁺ T cells (FIG. 11). Taken together, these data indicate that a FLT3L+TLR8L combination results in secretion of IL12 by dendritic cells, which will enhance intracellular Tbet expression within primed CD8+ T cells so that these cells display potent functional attributes.

### EXAMPLE 5

### A Combination of FLT3L and TLR8L Primes Qualitatively Superior Human CD8+ T-Cell Responses

Dendritic cells (DCs) govern the nature of de novo CD8⁺ T-cell responses primed from naïve precursors via the provision of processed antigens in the form of surface peptide-major histocompatibility complex (pMHC) class I molecules and other important signals, including costimulatory interactions and inflammatory cytokines. Much effort has therefore focused on the modulation of DC function to optimize CD8⁺ T-cell immunity⁶. The use of vaccine adjuvants, such as Toll-like receptor (TLR) ligands, can improve the immunogenicity of soluble protein and peptide antigens by mimicking pathogen-associated “danger” signals^{7, 8}. However, it is difficult to study the effects of potential adjuvants on human CD8⁺ T-cell responses due to the lack of a suitable model. Although the widespread use of murine systems has greatly advanced our knowledge of TLR function and DC/T-cell interactions, there are significant biological differences between mice and humans that complicate simple extrapolation between species. Moreover, traditional in vitro priming protocols that use human material rely on populations of inflammatory monocyte-derived DCs (moDCs) generated in a two-stage process from peripheral blood mononuclear cell (PBMC)-purified CD14⁺ monocytes⁹. In this setting, differentiation is achieved using a combination of GM-CSF and IL-4 prior to maturation with a cocktail of inflammatory cytokines or lipopolysaccharide (LPS)^{5, 10}. Although adequate for many purposes, this strategy has a number of limitations. In particular, the initial preparation of moDCs is laborious and time consuming. More importantly, no attempt is made to evaluate the role of conventional DCs and other resident blood cells (e.g. CD4⁺ T-cells and NK cells) in the priming process. To circumvent these drawbacks, we developed an innovative model of human CD8⁺ T-cell priming. This original in vitro approach is based on the rapid mobilization of DCs directly from unfractionated PBMCs (summarized in FIG. 1), enabling side-by-side comparisons of multiple test parameters in a standardized system with quantitative and qualitative readouts of the primed antigen-specific CD8⁺ T-cell response.

As circulating human DCs are rare, precursors within the starting PBMC material were mobilized using GM-CSF/IL-4 and matured with a standard cocktail of inflammatory cytokines (TNF, IL-1β, PGE2 and IL-7). Alternatively, mobilization was achieved by exposure to FLT3 ligand (FLT3L), which has demonstrable efficacy in animal studies¹¹. GM-CSF/IL-4 and FLT3L act on various immune cell subsets and mobilize distinct populations of DCs¹²⁻¹⁴. To ensure that sufficient numbers of antigen-specific CD8⁺ T-cells were present in the naïve pool, we restricted our analysis to the Melan-A/MART-1 epitope EAAGIGILTV (residues 26-35), which is recognized at remarkably high precursor frequencies in HLA-A*0201⁺ (HLA-A2⁺) individuals^{15, 16}. Moreover, to ensure optimal immunogenicity, we used the heteroclitic sequence ELAGIGILTV (ELA)¹⁷. The ELA epitope was incorporated into a 20mer synthetic long peptide (SLP, ELA-20) as a means of limiting antigen display to DCs with cross-presentation capacity¹⁸⁻²⁰. Prior to experimentation, we verified that the ELA-20 peptide required active processing. Specifically, we showed that ELA-20 was not presented directly by HLA-A2 and was not subjected to non-specific cleavage by enzymes present in serum.

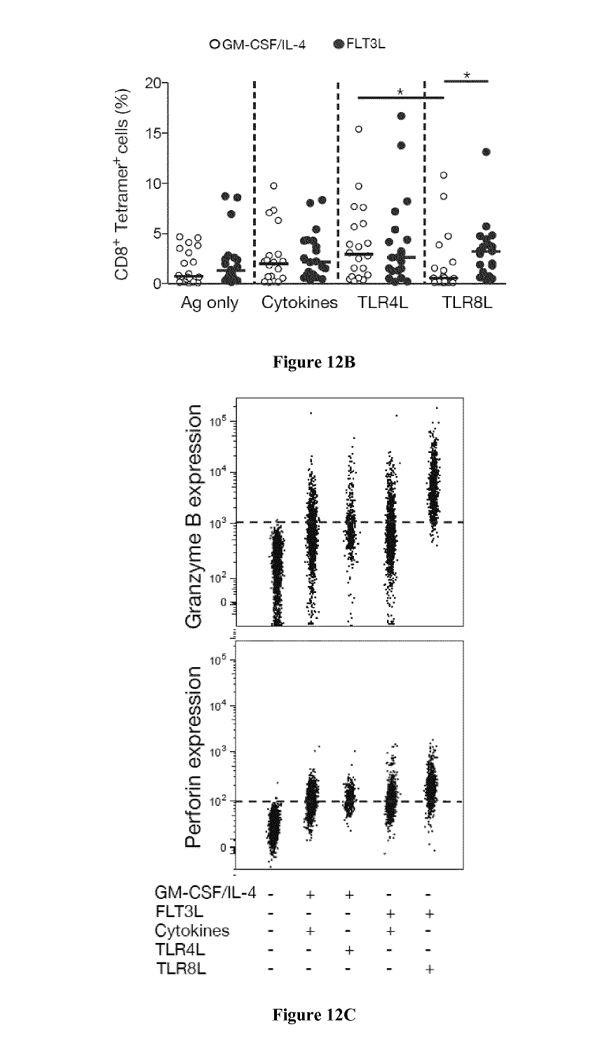

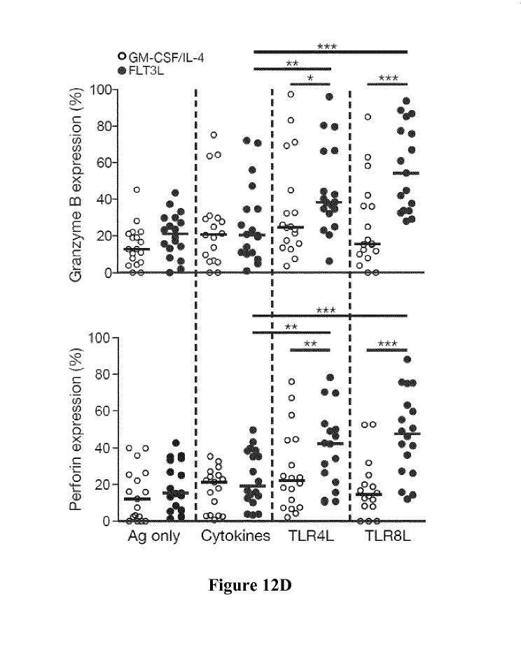

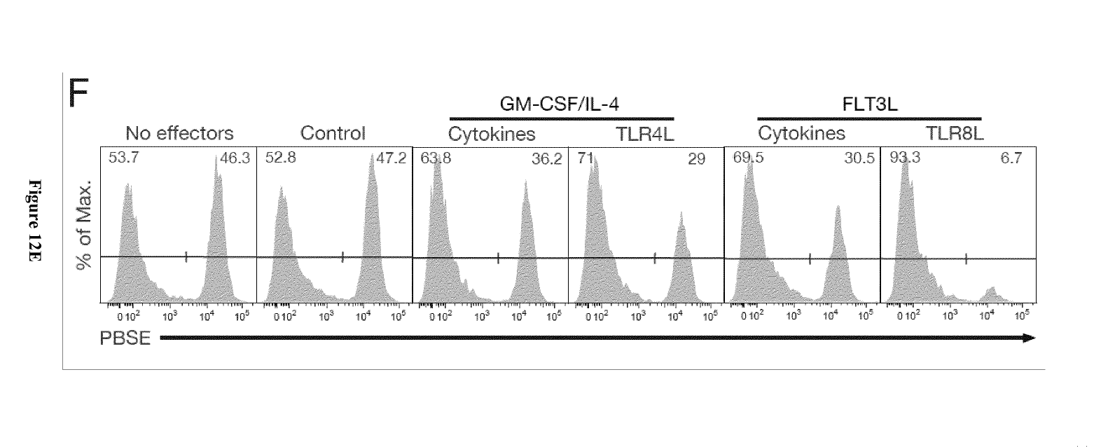

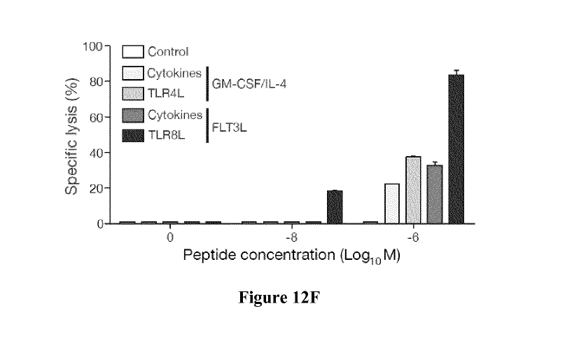

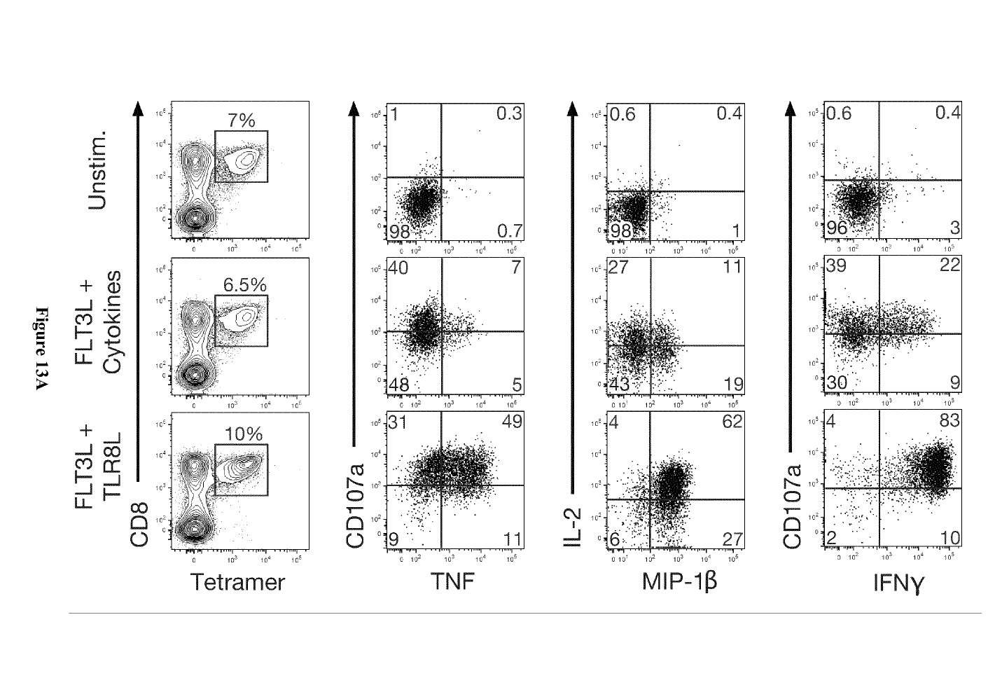

Next, we evaluated the optimal parameters for ELA-specific CD8⁺ T-cell priming in our system. An ELA-20 concentration of 1 μM consistently generated sufficiently large populations of primed cells for functional characterization (data not shown) and was therefore chosen for all downstream assays. Primed ELA-specific CD8⁺ T-cells peaked on day 10 (FIG. 12A), following identical kinetics and achieving comparable magnitudes with both the GM-CSF/IL-4 and FLT3L protocols. Subsequent experiments were therefore conducted over this time frame. We also confirmed that primed ELA-specific CD8⁺ T-cells originated from the naïve (CCR7⁺ CD45RA⁺) pool. After priming, the vast majority of ELA/HLA-A2 tetramer⁺ CD8⁺ T-cells displayed phenotypic hallmarks of antigen-driven expansion, predominantly differentiating into the effector memory (CCR7⁻ CD45RA⁻) compartment. No differences in terms of differentiation status were observed between ELA-specific CD8⁺ T-cell populations primed with either GM-CSF/IL-4 or FLT3L.

In subsequent experiments, we used our validated system to compare conventional adjuvant combinations alongside the largely uncharacterized ssRNA40 TLR8 ligand (TLR8L). The cellular distribution of TLR8 is entirely different between humans and mice, and is considered to be non-functional in the latter²¹. Moreover, TLR8-selective agonists²² have only recently become available²³. As a consequence, the adjuvant properties of TLR8L have not been fully assessed. The comparator in these assays was LPS, an extensively studied TLR4 ligand (TLR4L). Either TLR4L or TLR8L was introduced during the DC maturation step, in lieu of the inflammatory cytokine cocktail. Tetramer-based enumeration of ELA-specific CD8⁺ T-cell populations expanded in parallel revealed no major differences in the magnitude of priming across the three maturation conditions tested (FIG. 12B). However, the GM-CSF/IL-4/TLR8L combination primed significantly fewer ELA-specific CD8⁺ T-cells compared to the FLT3L/TLR8L combination, highlighting differences in the way that GM-CSF/IL-4 and FLT3L modulate subsets of antigen-presenting cells.

Notably, the FLT3L/TLR8L combination primed ELA-specific CD8⁺ T-cells endowed with significantly greater cytotoxic potential, as assessed by the expression of granzyme B and perforin (FIGS. 12C and 12D). Direct functional characterization confirmed this observation. In antigen-presenting cell lysis assays, FLT3L/TLR8L-primed CD8⁺ T-cells killed more than twice as many target cells presenting ELA-10 at a concentration of 1 μM compared with CD8⁺ T-cells primed under any other condition (FIGS. 12E and 12F). Moreover, only FLT3L/TLR8L-primed CD8⁺ T-cells were capable of eliminating targets presenting ELA-10 at a concentration of 10 nM.

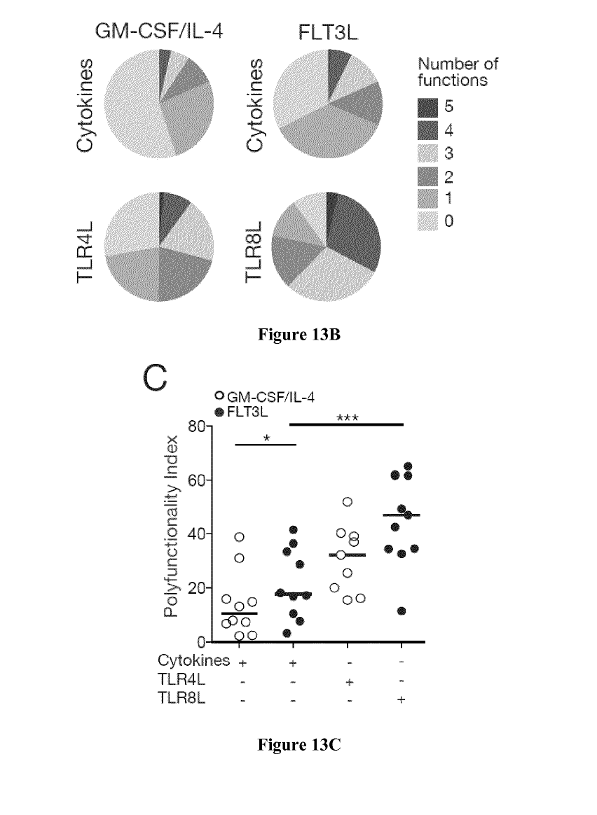

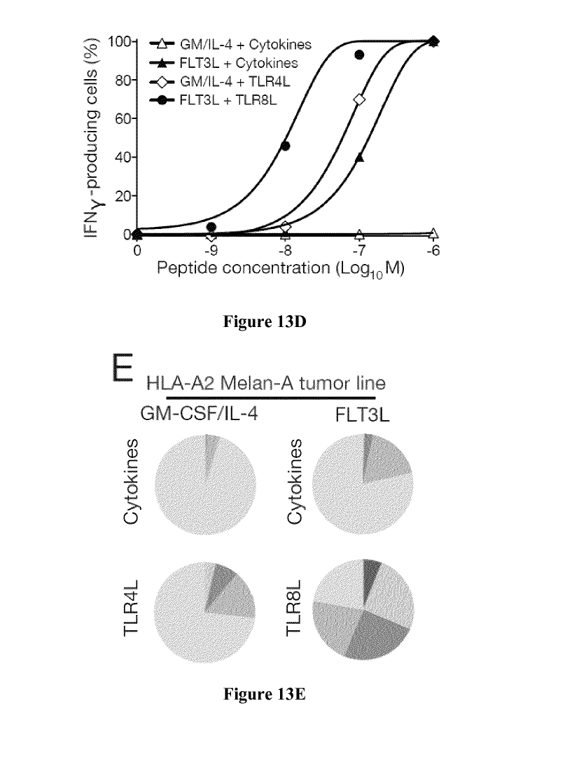

Next, we assessed the ability of primed CD8⁺ T-cells to deploy multiple effector functions in response to antigen encounter. In these assays, we measured the simultaneous induction of IFN-γ, MIP-1β, TNF and IL-2 together with surface mobilization of CD107a (FIG. 13A). Although GM-CSF/IL-4 and FLT3L in conjunction with the cytokine cocktail generated ELA-primed CD8⁺ T-cells expressing similar levels of cytotoxic molecules (FIGS. 12C and 12D), FLT3L treatment elicited greater frequencies of polyfunctional cells compared to GM-CSF/IL-4 (FIG. 13B). Moreover, ELA-specific CD8⁺ T-cells primed using the FLT3L/TLR8L combination displayed the highest levels of polyfunctionality (FIG. 13B). These differences were statistically significant in terms of the calculated polyfunctionality index across individual PBMC donors (FIG. 13C).

Antigen sensitivity (AgS) was evaluated via cognate peptide titration in IFN-γ secretion assays. FLT3L/TLR8L-primed ELA-specific CD8⁺ T-cells displayed the highest AgS, with a typical EC50 value (7.45×10⁻⁹ M) approximately one or two orders of magnitude lower compared to the GM-CSF/IL-4/TLR4L (5.77×10⁻⁸M) or FLT3L/cytokine (1.36×10⁻⁷M) combinations, respectively (FIG. 13D). IFN-γ production by GM-CSF/IL-4/cytokine-primed ELA-specific CD8⁺ T-cells was negligible. These differences in AgS between conditions extended across the full spectrum of effector functions, enabling FLT3L/TLR8L-primed CD8⁺ T-cells to deploy multiple effector functions at ELA-10 peptide concentrations as low as 1 nM. Crucially, FLT3L/TLR8L-primed ELA-specific CD8⁺ T-cells were also capable of mounting polyfunctional responses to an HLA-A2⁺ melanoma cell line presenting physiological levels of the endogenous Melan-A/MART-1 antigen. In contrast, the same tumor cell line was not recognized efficiently by ELA-specific CD8⁺ T-cells primed under other test conditions (FIG. 13E).

Next, we explored the mechanisms involved in the acquisition of superior functional attributes by FLT3/TLR8L-primed CD8⁺ T-cells. It is established that T-cell receptor (TCR) avidity, defined as the collective affinities of multiple monomeric TCR/pMHC interactions, can greatly influence AgS and thereby dictate the functional profile of CD8⁺ T-cells in response to cognate antigen^{24, 25}. Accordingly, we used wildtype and CD8-null ELA/HLA-A2 tetramers in parallel to quantify this parameter across different priming conditions²⁶. No significant differences in TCR avidity were detected between ELA-specific CD8⁺ T-cells primed with GM-CSF/IL-4 or FLT3 in conjunction with the cytokine cocktail or TLR ligands.

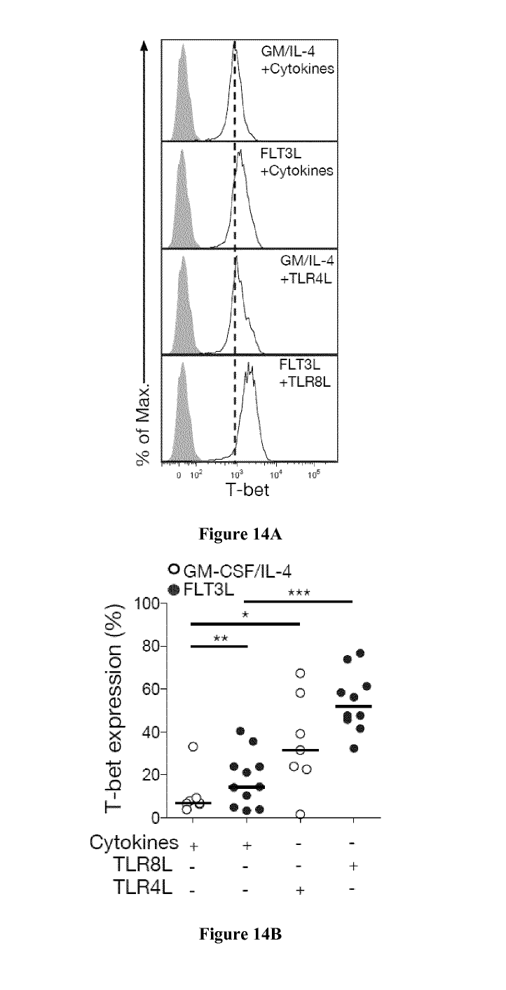

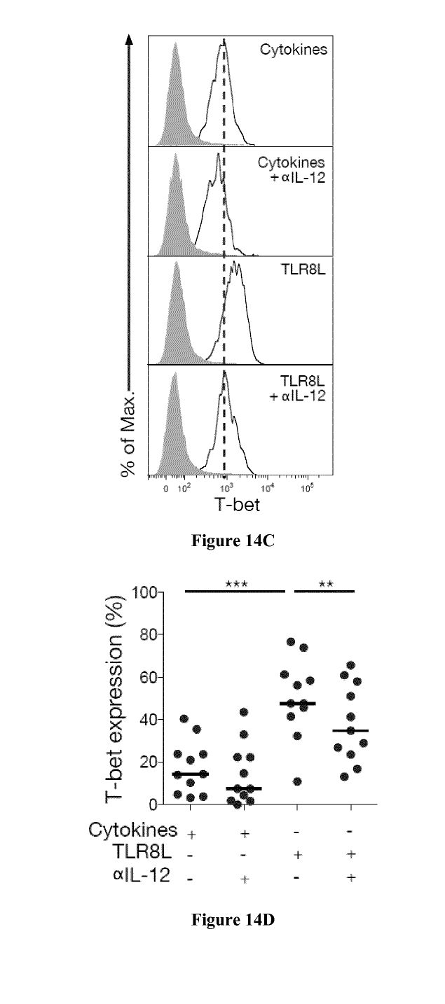

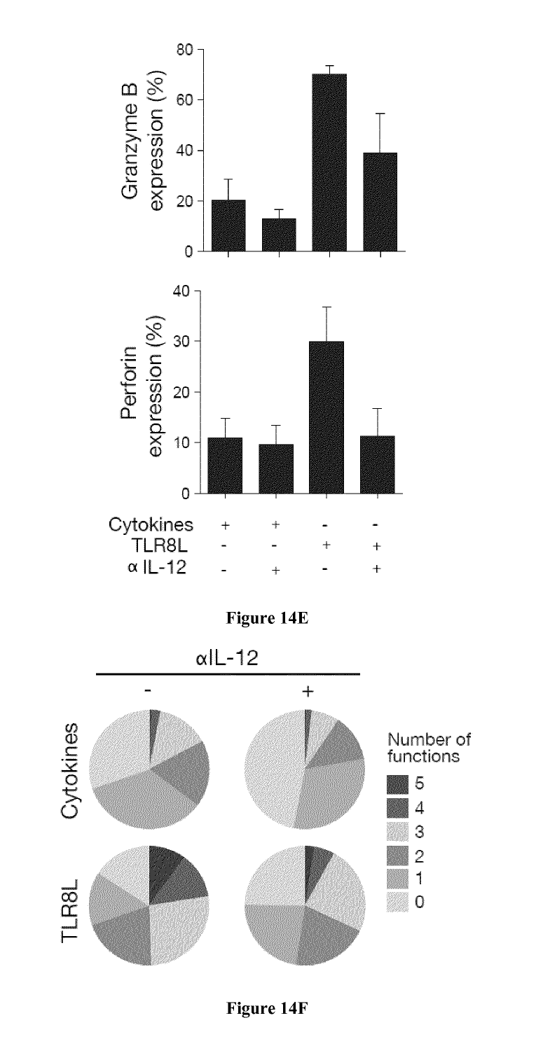

The expression of effector molecules such as granzyme B, perforin and IFN-γ is tightly controlled in CD8⁺ T-cells by the T-box transcription factor T-bet (also known as Tbx-21)²⁷. We detected significantly higher T-bet expression in ELA-specific CD8⁺ T-cells primed with FLT3L/TLR8L compared to other combinations (FIGS. 14A and 14B). T-bet expression is regulated by IL-12²⁸, which is secreted by myeloid DCs upon TLR8 ligation²². To assess the relationship between IL-12 levels and T-bet induction in our in vitro priming system, an α-IL-12p70 blocking antibody was administered daily during the first three days of culture. This intervention led to a significant drop in T-bet levels in FLT3L/TLR8L-primed CD8⁺ T-cells (FIGS. 14C and 14D). Moreover, ELA-specific CD8⁺ T-cells primed in the presence of the α-IL-12p70 blocking antibody expressed considerably less granzyme B and perforin (FIG. 14E), and were also less polyfunctional (FIG. 14F). Collectively, these data indicate that TLR8L, through effects on FLT3L-mobilized DCs and IL-12 production, triggers T-bet expression in primed CD8⁺ T-cells. In turn, T-bet endows these CD8⁺ T-cells with robust cytolytic machinery and superior AgS, most likely by reducing the TCR/pMHC activation thresholds required to trigger effector functions during cognate antigen engagement.

Our results demonstrate for the first time that a selective TLR8 agonist can act as an adjuvant to prime functionally superior antigen-specific CD8⁺ T-cells from human PBMCs. This advance was enabled by the development of an original in vitro priming model that offers several practical and theoretical advantages over existing systems.

In addition to streamlining the search for more effective adjuvants, our approach is applicable to several notable challenges in the field. For instance, the use of unmanipulated PBMCs will likely aid the identification of immunization regimens suitable for individuals at the extremes of age or those with advanced HIV infection, who are typically refractory to priming with standard vaccines. Our model could also facilitate the selection of antigen formulations best suited to the induction of highly functional de novo T-cell responses. The use of SLPs, which display several beneficial features for CD8⁺ T-cell priming compared to short peptides^{19, 20, 29, 30}, serves as one such example. Finally, the in vitro priming method presented here may expedite the generation of potent T-cells for adoptive therapy. It is notable in this respect that FLT3L/TLR8L-primed Melan-A/MART-1-specific CD8⁺ T-cells recognized naturally presented antigen on a tumor cell line and exhibited properties associated with in vivo efficacy. These findings are directly relevant to melanoma immunotherapy.

### REFERENCES

1. Appay, V., Douek, D. C. & Price, D. A. CD8+ T cell efficacy in vaccination and disease. Nat Med 14, 623-628 (2008).

2. Seder, R. A., Darrah, P. A. & Roederer, M. T-cell quality in memory and protection: implications for vaccine design. Nat Rev Immunol 8, 247-258 (2008).

3. Rosenberg, S. A., Yang, J. C. & Restifo, N. P. Cancer immunotherapy: moving beyond current vaccines. Nat Med 10, 909-915 (2004).

4. Pulendran, B. & Ahmed, R. Immunological mechanisms of vaccination. Nat Immunol 12, 509-517 (2011).

5. Martinuzzi, E. et al. acDCs enhance human antigen-specific T-cell responses. Blood 118, 2128-2137 (2011).

6. Banchereau, J. & Palucka, A. K. Dendritic cells as therapeutic vaccines against cancer. Nat Rev Immunol 5, 296-306 (2005).

7. Kawai, T. & Akira, S. The role of pattern-recognition receptors in innate immunity: update on Toll-like receptors. Nat Immunol 11, 373-384 (2010).

8. Coffman, R. L., Sher, A. & Seder, R. A. Vaccine adjuvants: putting innate immunity to work. Immunity 33, 492-503 (2010).

9. Sallusto, F. & Lanzavecchia, A. Efficient presentation of soluble antigen by cultured human dendritic cells is maintained by granulocyte/macrophage colony-stimulating factor plus interleukin 4 and downregulated by tumor necrosis factor alpha. J Exp Med 179, 1109-1118 (1994).

10. Wolff, M. & Greenberg, P. D. Antigen-specific activation and cytokine-facilitated expansion of naive, human CD8+ T cells. Nature protocols 9, 950-966 (2014).

11. Guermonprez, P. et al. Inflammatory Flt31 is essential to mobilize dendritic cells and for T cell responses during Plasmodium infection. Nat Med 19, 730-738 (2013).

12. Pulendran, B. et al. Flt3-ligand and granulocyte colony-stimulating factor mobilize distinct human dendritic cell subsets in vivo. J Immunol 165, 566-572 (2000).

13. Parajuli, P. et al. Flt3 ligand and granulocyte-macrophage colony-stimulating factor preferentially expand and stimulate different dendritic and T-cell subsets. Exp Hematol 29, 1185-1193 (2001).

14. Xu, Y., Zhan, Y., Lew, A. M., Naik, S. H. & Kershaw, M. H. Differential development of murine dendritic cells by GM-CSF versus Flt3 ligand has implications for inflammation and trafficking J Immunol 179, 7577-7584 (2007).

15. Pittet, M. J. et al. High frequencies of naive Melan-A/MART-1-specific CD8(+) T cells in a large proportion of human histocompatibility leukocyte antigen (HLA)-A2 individuals. J Exp Med 190, 705-715 (1999).

16. Dutoit, V. et al. Degeneracy of antigen recognition as the molecular basis for the high frequency of naive A2/Melan-a peptide multimer(+) CD8(+) T cells in humans. J Exp Med 196, 207-216 (2002).

17. Romero, P. et al. Antigenicity and immunogenicity of Melan-A/MART-1 derived peptides as targets for tumor reactive CTL in human melanoma. Immunol Rev 188, 81-96 (2002).

18. Joffre, O. P., Segura, E., Savina, A. & Amigorena, S. Cross-presentation by dendritic cells. Nat Rev Immunol 12, 557-569 (2012).

19. Chauvin, J. M. et al. HLA anchor optimization of the melan-A-HLA-A2 epitope within a long peptide is required for efficient cross-priming of human tumor-reactive T cells. J Immunol 188, 2102-2110 (2012).

20. Rosalia, R. A. et al. Dendritic cells process synthetic long peptides better than whole protein, improving antigen presentation and T-cell activation. Eur J Immunol 43, 2554-2565 (2013).

21. Jurk, M. et al. Human TLR7 or TLR8 independently confer responsiveness to the antiviral compound R-848. Nat Immunol 3, 499 (2002).

22. Gorden, K. B. et al. Synthetic TLR agonists reveal functional differences between human TLR7 and TLR8. J Immunol 174, 1259-1268 (2005).

23. Steinhagen, F., Kinjo, T., Bode, C. & Klinman, D. M. TLR-based immune adjuvants. Vaccine 29, 3341-3355 (2011).

24. Almeida, J. R. et al. Antigen sensitivity is a major determinant of CD8+ T-cell polyfunctionality and HIV-suppressive activity. Blood 113, 6351-6360 (2009).

25. Iglesias, M. C. et al. Escape from highly effective public CD8+ T-cell clonotypes by HIV. Blood 118, 2138-2149 (2011).

26. Price, D. A. et al. Avidity for antigen shapes clonal dominance in CD8+ T cell populations specific for persistent DNA viruses. J Exp Med 202, 1349-1361 (2005).

27. Glimcher, L. H., Townsend, M. J., Sullivan, B. M. & Lord, G. M. Recent developments in the transcriptional regulation of cytolytic effector cells. Nat Rev Immunol 4, 900-911 (2004).

28. Takemoto, N., Intlekofer, A. M., Northrup, J. T., Wherry, E. J. & Reiner, S. L. Cutting Edge: IL-12 inversely regulates T-bet and eomesodermin expression during pathogen-induced CD8+ T cell differentiation. J Immunol 177, 7515-7519 (2006).

29. Bijker, M. S. et al. CD8+ CTL priming by exact peptide epitopes in incomplete Freund's adjuvant induces a vanishing CTL response, whereas long peptides induce sustained CTL reactivity. J Immunol 179, 5033-5040 (2007).

30. Melief, C. J. & van der Burg, S. H. Immunotherapy of established (pre)malignant disease by synthetic long peptide vaccines. Nat Rev Cancer 8, 351-360 (2008).

### Example 6

### Impaired Naïve CD8⁺ T Cell Priming in Elderly Individuals or HIV Infected Patients

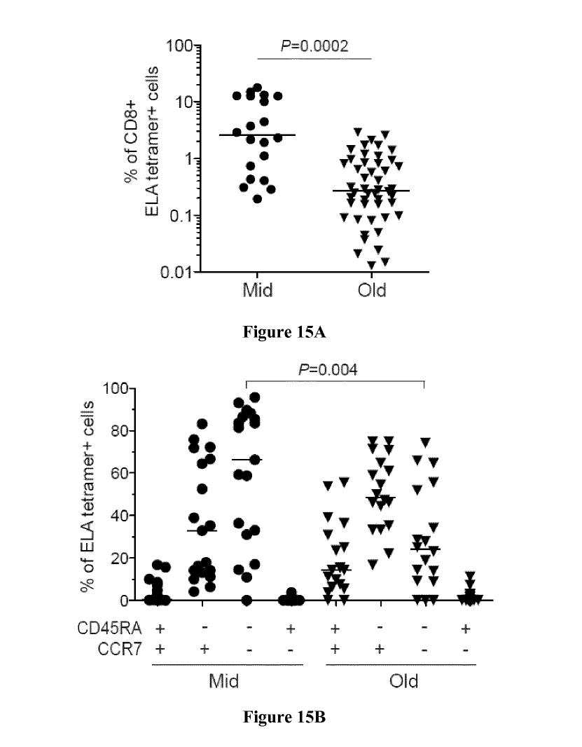

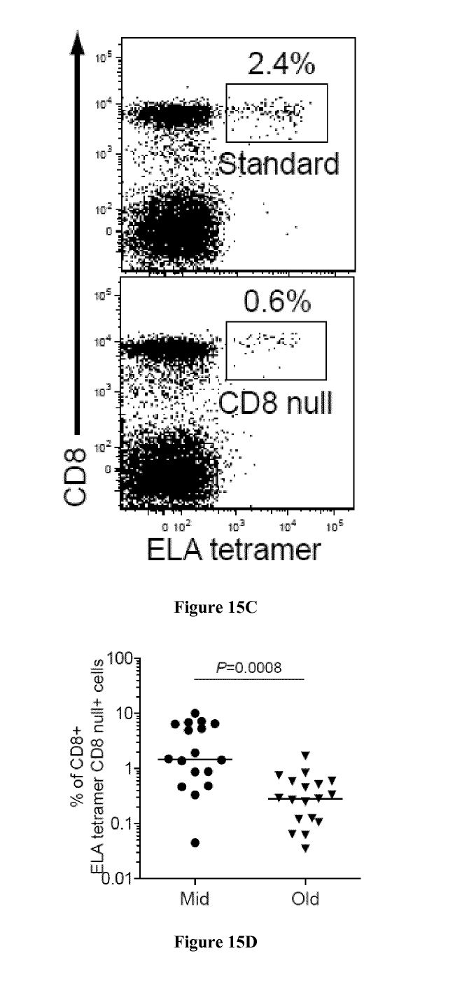

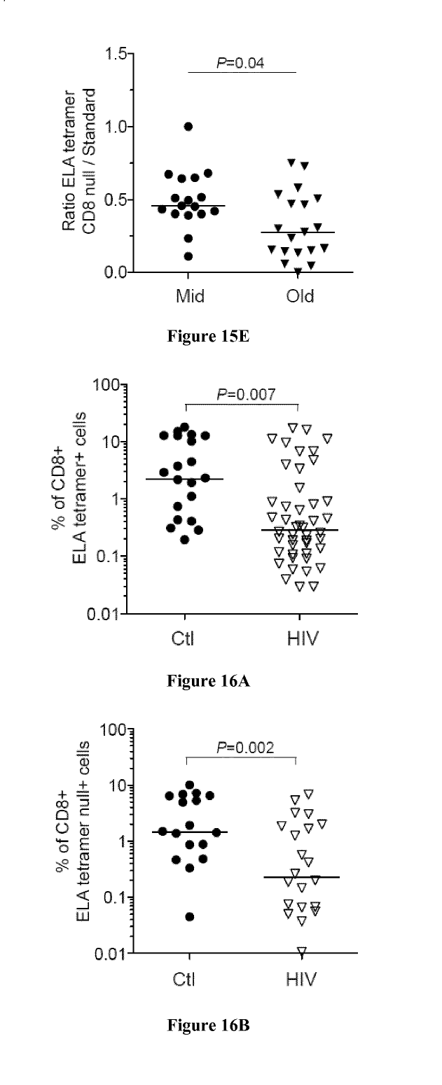

The magnitude of ELA/HLA-A*02:01 tetramer⁺ cells after in vitro expansion was used to assess antigenspecific CD8⁺ T cell priming capacity in healthy middle-aged adults and healthy elderly (>70 years old) adults. Using this approach, we found that ELA-specific CD8⁺ T cell expansion was significantly lower in elderly individuals compared to healthy middle-aged controls (FIG. 15A). This indicates that advanced age is associated with quantitatively impaired CD8⁺ T cell priming. Moreover, while the vast majority of primed ELA-specific CD8⁺ T cells expressed a CD45RA⁻ CCR7^{+/-} memory phenotype, expanded tetramer⁺ cells from old donor PBMCs were usually less differentiated (presenting fewer CD45RA⁻ CCR7⁻ cells) compared to cells expanded from middle-aged donors (FIG. 15B). This suggests a less efficient activation of elderly naïve CD8⁺ T cells, and differentiation into effector memory cells, potentially due to qualitative cellular defects, which is reminiscent of recent observations from an aged mouse model of infection (Smithey et al. 2011. Eur J Immunol 41, 1352-64).

In donors with large expansions of ELA-specific CD8⁺ T cells after in vitro priming (i.e. >0.4% ELA/HLA-A*02:01 tetramer CD8⁺ T cells), we performed parallel experiments using CD8-null ELA/HLA-A*02:01 tetramers (FIG. 15C). These reagents incorporate the compound D227K/T228A mutation in the α3 domain of the HLA-A*02:01 protein that abrogates CD8 coreceptor binding, thereby enabling the selective identification of high avidity antigen-specific CD8⁺ T cells (Price et al. 2005. J Exp Med 202, 1349-6; and Wooldridge et al. 2009. Immunology 126, 147-64), which are known to display superior functional (e.g. cytolytic and polyfunctional) attributes and greater efficacy against pathogens and tumors (Appay et al. 2008. Nat Med 14, 623-8). In line with the results obtained using standard tetramers, significantly lower frequencies of primed CD8-null ELA/HLA-A*02:01 tetramer CD8⁺ T cells were observed in elderly individuals compared to middle-aged controls (FIG. 15D). Importantly, the CD8-null/standard tetramer frequency ratios were also lower in the elderly group (FIG. 15E). Thus, advanced age is associated with lower frequencies of avidity-impaired primary responses, presumably as a result of repertoire perturbations within the naïve CD8⁺ T cell pool. This is in line with a recent report of increased proportions of low avidity cells within virus specific memory CD8⁺ T cells in old individuals (Griffiths et al. 2013. J Immuno1190, 5363-72).

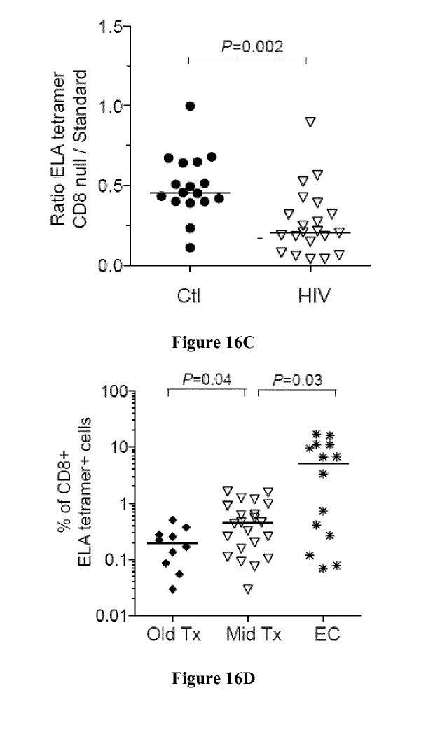

Parallel investigations of CD8+ T cell priming efficacy were performed in HIV infected donors. For this purpose, we selected treated (i.e. progressors) or untreated (i.e. non progressors or elite controllers) HIV infected patients with undetectable viral load to avoid potential bias or influence of HIV replication and elevated inflammation on the assessment of T cell priming efficacy. We observed that ELA-specific CD8⁺ T cell expansion was significantly lower in HIV infected patients compared to healthy controls (FIG. 16A). Moreover, like for the elderly, significantly lower frequencies of primed CD8-null ELA/HLA-A*02:01 tetramer⁺ CD8⁺ T cells (FIG. 16B), as well as CD8-null/standard tetramer frequency ratios (FIG. 16C), were found in HIV infected patients compared to the controls. Of note, the ELA-specific CD8⁺ T cell expansion differed according to the progression status and age of HIV infected donors. For instance, elite controllers (who control HIV replication naturally) displayed a significantly higher CD8+ T cell priming efficacy compared to age matched treated patients that were progressing towards the disease, who themselves showed higher priming efficacy than old (>65 y old) treated patients (FIG. 16D).

### Example 7

### Importance of the Naïve T Cell Compartment for Effective Priming

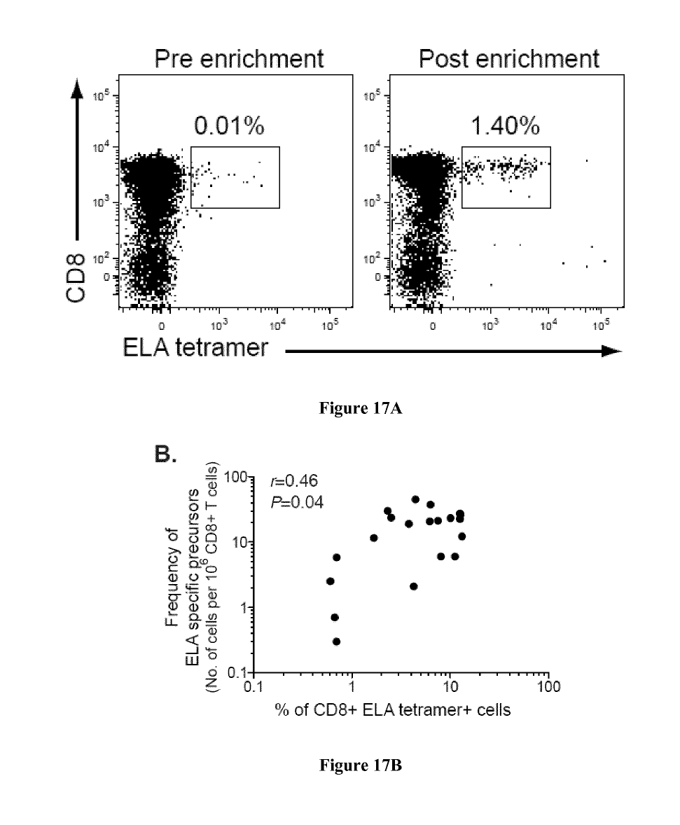

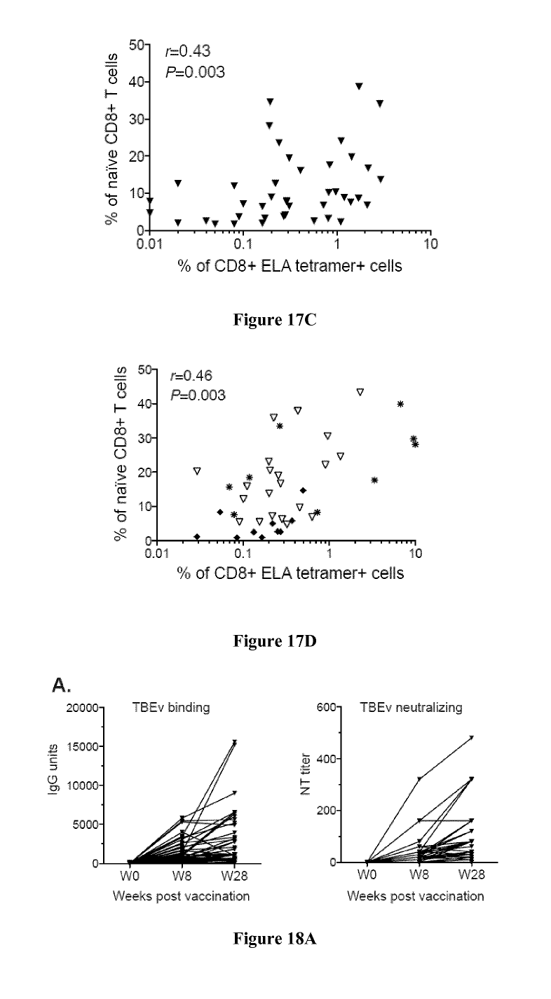

Recent studies in murine models suggest that the frequency of naïve T cell precursors correlates with the magnitude of the primary T cell response (Moon et al. 2007. Immunity 27, 203-13; Obar et al. 2008. Immunity 28, 859-69; and Kotturi et al. 2008. J Immunol 181, 2124-33). Accordingly, we quantified naïve ELA-specific CD8⁺ T cell precursor frequencies in a subset of healthy donors using an established procedure for the enrichment of CD45RA⁺ CCR7⁺ tetramer⁺ cells from PBMC samples via magnetic separation (FIG. 17A). A direct correlation was observed between the frequency of ELA/HLA-A*0201 tetramer cells after in vitro expansion and the frequency of ELA-specific CD8⁺ T cell precursors (FIG. 17B). Due to the high number of PBMCs required for antigen specific precursor quantification, the same approach was not possible in elderly individuals. Instead, we measured the frequency of total naïve (CD45RA⁺ CCR7⁺ CD27⁺) CD8⁺ T cells in these donors. A direct correlation was observed between the frequency of primed ELA/HLA-A*0201 tetramer cells and the frequency of naïve CD8⁺ T cells in this group (FIG. 17C). Moreover, we found a similar correlation between the frequency of primed ELA/HLA-A*0201 tetramer⁺ cells and the frequency of naïve CD8⁺ T cells in HIV infected donors (FIG. 17D). Overall, the present data support a relationship between the size of the naïve T cell repertoire and the efficacy of CD8⁺ T cell priming in humans. Accordingly, impaired CD8⁺ T cell priming in the elderly or the HIV infected population can reasonably be attributed to reduced thymic output, disturbed homeostatic maintenance and a consequent reduction in naïve T cell frequencies.

### Example 8

### Relationship Between the Induction of De Novo Immune Responses In Vitro and In Vivo

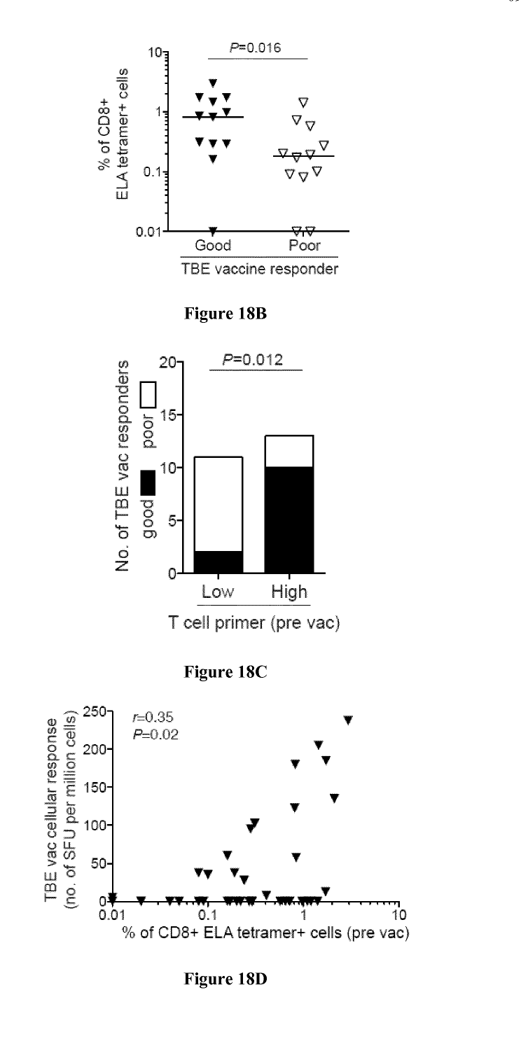

We also studied forty elderly individuals (>70 years) who were vaccinated for the first time against tick-borne encephalitis virus (TBEv). The individuals selected for this study had never been exposed to TBEv beforehand. De novo humoral and cellular immune responses to TBEv vaccination were monitored at weeks 8 and 28 or 26 post-immunization respectively, and compared to baseline values. Among these vaccinees, we defined good and poor TBEv vaccine responders as donors with both TBEv binding and neutralizing antibody levels at weeks 8 or 28 post-immunization within the top and bottom quartiles of all titer values, respectively (FIG. 18A). Good TBE vaccine responders displayed significantly stronger CD8⁺ T cell priming efficacies in vitro compared to poor responders (FIG. 18B). Moreover, the frequency of ELA/HLA-A*0201 tetramer⁻ cells after in vitro expansion assessed at day 0 (i.e. pre-vaccination) was associated with subsequent TBE vaccine responsiveness. High primers with ELA/HLA-A*0201 tetramer cell expansions above the median frequency of 0.28% at day 0 constituted a significantly greater proportion of good TBE vaccine responders compared to low primers (FIG. 18C). In addition, we found a direct correlation between in vitro CD8⁺ T cell priming capacity at day 0 and ex vivo TBE cellular responses measured at week 26 post-immunization (FIG. 18D). These data indicate that CD8⁺ T cell priming efficacy as measured in vitro is representative and can even predict the subsequent in vivo primary response to vaccination in the elderly.

### REFERENCES

Throughout this application, various references describe the state of the art to which this invention pertains. The disclosures of these references are hereby incorporated by reference into the present disclosure.

1. An in vitro method for testing T cell priming efficacy in a subject comprising the steps of a) providing a sample from the subject, b) culturing the sample in a medium which induces differentiation of dendritic cells, c) maturing the dendritic cells obtained at step a) in presence of an amount of at least one antigen and an amount of at least one cytokine or at least one ligand suitable for the activation of a pathogen recognition receptor, d) priming and expanding T cells present in the sample and e) analyzing the polyfunctionality of primed T cells obtained at step d).

2. The method of claim 1 wherein CD4+ T cell priming efficacy is tested.

3. The method of claim 1 wherein CD8+ T cell priming efficacy is tested.

4. The method of claim 1 wherein the subject harbors one or more HLA Class I alleles and one or more HLA Class II alleles.

5. The method of claim 1 wherein the sample is a biopsy sample.

6. The method of claim 1 wherein the sample is a peripheral blood mononuclear cell (PBMC) sample.

7. The method of claim 1 wherein the culture medium comprises Granulocyte/Macrophage Colony-Stimulating Factor (GM-CSF) and interleukin 4 (IL-4).

8. The method of claim 1 wherein the culture medium comprises FMS-like tyrosine kinase 3 (Flt-3) ligand.

9. The method of claim 1 wherein the culture medium comprises IL-1beta.

10. The method of claim 1 wherein the at least one cytokine is selected from the group consisting of IL1-beta, IL-7, interferons, and TNF-alpha.

11. The method of claim 1 wherein the at least one ligand that is suitable for the activation of a pathogen recognition receptor is a Toll-like receptor (TLR) agonist.

12. The method of claim 11 wherein the TLR agonist is selected from the group consisting of TLR1, TLR2, TLR3, TLR4, TLR5, TLR6, TLR7, TLR8, TLR9, TLR10, TLR11, TLR12, and TLR13 agonists

13. The method of claim 1 wherein the at least one ligand that is suitable for the activation of a pathogen recognition receptor is a NOD-like receptor ligand.

14. The method of claim 1 wherein the at least one antigen is selected from the group consisting of viral antigens, bacterial antigens, fungal antigens, and cancer-associated antigens.

15. The method of claim 1 wherein the at least one antigen is a MHC-class I restricted antigen.

16. The method of claim 1 wherein the at least one antigen is a HLA-A2 restricted antigen.

17. The method of claim 1 wherein the at least one antigen is SEQ ID NO:1 or 2.

18. The method of claim 1 wherein step d) is performed by adding fetal calf serum (FCS) or fetal bovine serum (FBS) to the culture medium.

19-20. (canceled)

21. A method for screening a test substance for its adjuvant properties comprising the steps of
i) obtaining primed T cells by
a) providing a sample from a subject,
b) culturing the sample in a medium which induces differentiation of dendritic cells,
c) maturing the dendritic cells obtained at step b) in the presence of at least one antigen, at least one cytokine or at least one ligand suitable for the activation of a pathogen recognition receptor, and the test substance,
d) priming and expanding T cells present in the sample,
ii) determining the polyfunctionality of the primed T cells as above described obtained in step d)
iii) comparing the polyfunctionality determined at step ii) with a predetermined reference polyfunctionality and
iv) selecting the test substance as an adjuvant when the polyfunctionality determined at step iii) is superior or equal to the predetermined reference polyfunctionality.

22. A method for predicting vaccine responsiveness of a subject comprising
a) providing a sample from the subject,
b) culturing the sample in a medium which induces differentiation of dendritic cells,
c) maturing the dendritic cells obtained at step b) in the presence of at least one antigen from the vaccine, at least one cytokine or at least one ligand suitable for the activation of a pathogen recognition receptor,
d) priming and expanding T cells present in the sample,
e) determining the polyfunctionality of the primed T cells obtained in step d)
f) comparing the polyfunctionality determined at step e) with a predetermined reference polyfunctionality and
g) concluding that the subject will respond effectively to the vaccine when the polyfunctionality determined at step iii) is superior or equal to the predetermined reference polyfunctionality.

23. A vaccine composition comprising at least one antigen, at least one FLT-3 ligand and at least one TLR8 agonist.

24. The vaccine composition of claim 23 wherein the antigen is a cancer antigen.

25. A method of treating cancer in a subject in need thereof, comprising
administering to the subject a vaccine composition comprising at least one antigen, at least one FLT-3 ligand and at least one TLR8 agonist

26. The method of claim 5, wherein the biopsy sample is a tumor sample.
